(19) United States
(12) Patent Application Publication
(10) Pub. No.: US 2025/0262146 A1
(43) Pub. Date: Aug. 21, 2025

(54) PLATFORM NANOPARTICLE TECHNOLOGY FOR SUSTAINED DELIVERY OF HYDROPHOBIC DRUGS

(71) Applicant: UNIVERSITY OF MARYLAND, BALTIMORE, Baltimore, MD (US)

(72) Inventors: Tao LOWE, Hummelstown, PA (US); Curt I. CIVIN, Baltimore, MD (US)

(21) Appl. No.: 18/859,226

(22) PCT Filed: Apr. 26, 2023
(86) PCT No.: PCT/US2023/019974

§ 371 Date: Oct. 23, 2024

Related U.S. Application Data
(60) Provisional application No. 63/334,938, filed on Apr. 26, 2022.
(60) Provisional application No. 63/334,896, filed on Apr. 26, 2022.

(51) Int. Cl. A61K 9/00; A61K 31/366; A61K 47/20; A61K 47/34; A61P 35/02; B82Y 5/00

(52) U.S. Cl. CPC A61K 9/0024; A61K 31/366; A61K 47/20; A61K 47/34; A61P 35/02; B82Y 5/00

(57) ABSTRACT

This invention relates to a nanogel platform technology that can increase the water solubility of hydrophobic compounds, particularly hydrophobic compounds having one or more double bonds, by more than about 400 fold. Methods according to embodiments of the invention involve copolymerizing the hydrophobic compound with N-isopropylacrylamide monomer and dextran-lactate-2-hydroxyethyl-methacrylate macromer via UV emulsion polymerization in aqueous solution. The resulting nanosystem has the additional advantage of being able to sustain the release of the drug candidate for a long period of time, thus increasing the half-life, long-term bioavailability and therapeutic effects of these hydrophobic compounds.

### CROSS-REFERENCE TO RELATED APPLICATIONS

This application is a 371 National Stage application of PCT Application No. PCT/US2023/019974, filed 26 Apr. 2023, which claims the benefit of U.S. provisional application Ser. No. 63/334,938, filed 26 Apr. 2022 and U.S. provisional application Ser. No. 63/334,896, filed 26 Apr. 2022. The entire contents of these applications are hereby incorporated by reference as if fully set forth herein.

### GOVERNMENT FUNDING SUPPORT

This invention was made with government support under grant no. EY023853, awarded by the National Institutes of Health. The government has certain rights in the invention.

### BACKGROUND

### 1. Field of the Invention

The invention relates to the general field of medicine and pharmaceutics, and in particular to a nanogel formulation for hydrophobic drugs which increases water solubility and half-life of the hydrophobic drugs and provides long-term bioavailability of the hydrophobic drugs to the subject in need of such treatment.

### 2. Background of the Invention

A drug must enter solution in order to achieve the desired concentrations in the systemic circulation and at the target site for the desired biological effect. More than 40% of newly developed chemical entities are hydrophobic and have very low water solubilities. Thus, low aqueous solubility is the major problem in new drug development. Various techniques have been used to moderate the water solubility of hydrophobic drugs, including physical methods of reducing the drug particle size via micronization and nanosuspension, crystal engineering, solid dispersion, use of surfactants, solubilizers and cosolvents, drug dispersion in carriers including liposomes, dendrimers and PLGA nanoparticles, solid solutions, cryogenic techniques and supercritical fluid; and chemical methods of changing pH, use of buffer, derivatization, salt formation, and complexation. However, for many hydrophobic drug candidates, none of these techniques sufficiently address their limited water solubility.

Artemisinins (ARTs), discovered in 1971 by Dr. Youyou Tu, is an extract from the sweet wormwood plant that has antileukemic effects. ART and its derivatives have been known as a useful drug for malaria treatment without toxicity. ART and many ART derivatives are hydrophobic. Artesunate is a derivative of artemisinin having a hydrophilic group, which makes it a more potent drug than other artemisinins and its derivatives.

Although the mechanism of action is not entirely known, it is generally thought that an iron-dependent activation of the endoperoxide bridge induces the generation of high levels of intracellular reactive oxygen species (ROS), which is responsible for the drug's antimalarial activity. Based on such cytotoxicity, ART and its derivatives also are known to have efficacy against cancer cells, including leukemia.

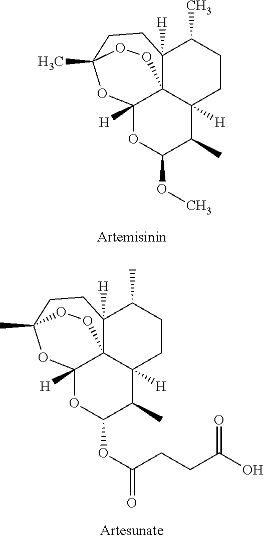

Leukemia is an umbrella term for various devastating blood cancers that affect all ages, with 470,000 and 61,000 newly diagnosed cases and about 300,000 and 23,000 deaths annually in the world and USA, respectively. The total cost for the treatment of leukemias in USA was $8.7B in 2015 and is expected to increase to $13B by 2030. Acute myeloid leukemia (AML) is a type of leukemia that is a cancer of bone marrow that results from impaired differentiation and proliferation of myeloid cells. This disease has a high death rate in the United States, with over 11,000 deaths so far in 2022, and increasing costs for treatment.

In recent years, targeted therapies have provided new options to manage acute leukemias. However, chemotherapy (monotherapy, the standard care, or targeted therapies) has a shallow response and resistance is developed due to the short half-life and water insolubility of the chemotherapeutic agents. Thus, the survival benefit is not ideal.

For the treatment of leukemia, recently a type of ART derivatives has been demonstrated as a potential therapeutic alternative to current chemotherapies. In particular, several 2-carbon linked dimeric artemisinin analogs (2C-ARTs (see U.S. Pat. No. 9,487,538) were found to effectively kill 9 human leukemia cell lines at half maximal inhibitory concentrations (IC₅₀)<50 nM.

In the art of pharmaceutics, in general, there is a need in the art for formulations that can deliver drugs that are hydrophobic, have short half-lives, or both. Such sustained delivery vehicles would be able to benefit patients being treated with any of these drug entities, which currently require frequent administrations, long infusion times, or both and are suitable for use with a variety of different pharmaceutical compounds. In particular, there is an unmet need in the art for a product and method that can increase the water solubility and half-lives of pharmaceutical compounds to provide long-term bioavailability of the drug for therapy and treatment in patients.

### SUMMARY OF THE INVENTION

Therefore, this invention provides a nanogel technology, composition and method that is useful to administer drugs to a patient in need, specifically by increasing the water solubility of hydrophobic compounds by more than 400 fold. It is particularly useful for drugs that contain one or more double bonds and are highly hydrophobic, because the drug itself becomes part of the polymer, but can be used for other drug compositions as well.

In particular embodiments, the present invention relates to a polymerized nanogel pharmaceutical composition, comprising: (a) a hydrophobic drug; (b) optionally sodium dodecyl sulfate (SDS); and (c) a biodegradable nanogel composition comprising (i) hydrolyzable macromer with two or more double bonds; (ii) a monomer; and (iii) an initiator, wherein the macromer, the monomer, and the hydrolysable crosslinker are reacted with an initiator to form a biodegradable nanogel in the presence of the hydrophobic drug.

In certain embodiments, the composition has a size of about 1 nm to 1000 nm or has a size of about 1 to 600 nm or has a size of about 10 to about 350 nm.

In preferred embodiments, the hydrophobic drug contains one or more double bonds, preferably a C═C double bond.

In certain embodiments, the hydrophobic drug is selected from the group consisting of artemisinin, an artemisinin derivative, ART631, artemisinin conjugated with other anticancer pharmacophores, 3-carbon-linked artemisinin-derived dimer (3C-ART), 2-carbon-linked dimeric artemisinin-derived analogs, osimertinib, sunitinib, quinine, lumefantrine, stiripentol, glecaprevir, cyclosporin, voclosporin, naloxone, betulinic acid, bevirimat, derivatives, dienestrol, neuroprotection, prostaglandin, unsaturated fatty acids, rilpivirine, polyene antimycotics, or a combination thereof. A preferred hydrophobic drug is ART631.

In certain embodiments, the hydrolysable macromer is dextran grafted with oligolactate-(2-hydroxyetheyl methacrylate) (Dex-PLA-HEMA), poly-ε-caprolactone-(2-hydroxyethyl methacrylate (Dex-PCL-HEMA), or a hydrolyzable molecule composed of dextran, polylactic acid, polylactic-co-glycolic acid, polyethylene glycol, poly(ethylene oxide)-poly(propylene oxide)-poly(ethylene oxide), poly(ethylene oxide)-co-poly(L-lactic acid), biotinylated poly(ethylene glycol-block-lactic acid), pluronic acid, polaxamer, polyesters, polyamides, poly(amino acid), polyurethane, polyorthoesters, polyanhydrides, polyethylene terephthalate, polycarbonates, polyfumarates, polycyanoacrylates, poly(alkylcyanoacrylate), polyphosphazenes, polyphosphoesters, or poly(bis(p-carboxyphenoxy) propane-sebacic acid), or a combination thereof, wherein the hydrolysable molecule contains one or more double bonds.

In certain embodiments, the monomer is selected from the group consisting of N-isopropylacrylamide, N-alkylacrylamide, N-n-propylacrylamide, N-isopropylmethacrylamide, or any combination thereof.

In certain embodiments, the initiator is selected from the group consisting of 2-hydroxy-4′-(2-hydroxyethoxy)-2-methylpropiophenone, 2,2′-azobis [2-(2-imidazolin-2-yl) propane]dihydrochloride, 1-hydroxycyclohexylphenyl-ketone, 2-hydroxy-2-methyl-1-phenylpropanone, 2,2-dimethoxy-1,2-diphenyl-ethan-1-one, 2-(4-Methylbenzyl)-2-(dimethylamino)-1-(4-morpholinophenyl) butan-1-one, alpha hydroxy ketones, phosphine oxides, benzophenone, thioxanthones, 2,2-dimethoxy-2-phenylacetophenone, isopropyl thioxanthone, 2-ethylhexyl-(4-N,N-dimethyl amino)benzoate, ethyl-4-(dimethylamino)benzoate, peroxides, benzoyl peroxide, molecular oxygen, azobisisobutyronitrile, camphorquinone, eosin Y, triethanolamine, 1-vinyl-2-pyrrolidinone, or a combination thereof.

In certain embodiments, hydrophobic drug is released from the nanogel for at least 50 days, at least 60 days, or at least 90 days.

In other embodiments, the invention relates to a method of drug delivery to a subject in need thereof comprising administering to the subject the nanogel pharmaceutical composition described herein. Preferably, the subject is suffering from a disease or conditions selected from the group consisting of cancer, malaria, fungus, infection, inflammation, seizure, stroke, depression, hepatitis C, diabetes, diabetic retinopathy, age-related macular degeneration, glaucoma, dry eye, Alzheimer's disease, Parkinson's disease, neurological disorders, pain, temporomandibular joint disorders, immune system disorders, opioid overdose or thereof. In preferred embodiments, is suffering from cancer, such as wherein the cancer is leukemic, skin, melanoma, lung, bronchus, kidney, liver, breast, oral, head, neck, esophageal, thyroid, eye, retinal, ear, bone, cartilage, fat, muscle, blood vessel, gastrointestinal stromal, intrahepatic bile duct, bladder, colon, rectum, vaginal, prostate, testicular, pancreatic, cervical, uterine, pleural, immune system, glioblastoma, Non-Hodgkin lymphoma, carcinoma-adenocarcinoma, basal cell carcinoma, squamous cell carcinoma and transitional cell carcinoma, sarcoma, lymphoma, myeloma, brain, or spinal cord cancer thereof. Most preferably, the cancer is a leukemia.

In certain embodiments, the nanogel pharmaceutical composition is administered by injection.

In certain embodiments, the invention relates to a method of increasing the solubility and effective pharmacologic half-life of a double bond-containing hydrophobic drug compound using a nanogel pharmaceutical composition of claim 1.

In certain embodiments, the invention relates to a method of making a nanogel pharmaceutical composition of claim 1, comprising: (a) dissolving a double bond containing hydrophobic drug compound in an organic solvent; (b) adding the hydrophobic drug to solution a solution of water optionally also containing SDS; (c) dissolving a macromer selected from the group consisting of dextran-polylactic acid-2-hydroxyethyl-methacrylate, dextran grafted oligolactate-(2-hydroxyethyl methacrylate), and poly-¿-caprolactone-(2-hydroxyethyl methacrylate (Dex-PCL-HEMA) macromer, in water also optionally containing SDS to form a solution; (d) combining the macromer solution and NIPAAM solution to the drug compound solution; (e) adding 2-hydroxy-4′-(2-hydroxyethyl)-2-methyl propiophenone to the combined mixture in water; (f) incubating the mixture in an oil bath with nitrogen purging; and (g) during nitrogen purging, curing the mixture solution by exposure to UV light for at a wavelength of 320 nm to 500 nm for about 2 minutes to about 5 hours.

### BRIEF SUMMARY OF THE DRAWINGS

FIG. 1 is a drawing showing the scheme for Art631 nanogel production.

FIG. 2 provides the chemical structure of the dextran-polylactic acid (DEX-PLA-HEMA) poly (N-isopropylacrylamamide) macromer.

FIG. 3 provides a drawing of the dextran-polylactic acid (DEX-PLA-HEMA) poly (N-isopropylacrylamamide) (pNIPAAM) nanogel.

FIG. 4A and FIG. 4B present Fourier-transform infrared (FTIR) spectra of plain PLA nanogels and plain PLA/SDS nanogels (FIG. 4A), and NanoART631 nanogels at the indicated wt % (FIG. 4B).

FIG. 5A presents 1H NMR spectra of ART631 with peak assignments. FIG. 5B provides the 1H NMR spectra of PLA/SDS nanogels alone, and 5 wt % NanoART631. FIG. 5C and FIG. 5D present 1H NMR spectra of physically mixed full amount ART631 and plain nanogels, and physically mixed half amount ART631 and plain nanogels, respectively.

FIG. 6A presents 1H NMR spectra of ART631 at concentrations 50, 100, 250, 500, 1000 and 10000 μg/mL. FIG. 6B, FIG. 6C, FIG. 6D, FIG. 6E, and FIG. 6F present graphs showing concentration calibration curves of ART631 at peaks 0.7, 0.8, 0.9, 1.27 and 1.3 ppm regions, as indicated.

FIG. 7A presents 1H NMR spectra of NanoART631 containing 2, 5, 10, 12.5 and 15 wt % ART631. FIG. 7B, FIG. 7C, FIG. 7D, FIG. 7E, and FIG. 7F are graphs showing correlation curves of measured and theoretical ART631 amounts loaded into the nanogels based on the ART631 characteristic peaks at chemical shifts 0.7, 0.8, 0.9, 1.27, and 1.3 ppm regions, as indicated.

FIG. 8 is a table that presents synthesis yield, encapsulation efficiency (EE %), loading content (LC %), hydrodynamic diameter, polydispersity index (PDI), zeta potential of nanogels containing 0, 2, 5, 7.5, 10, 12.5 and 15 wt % ART631 in DI water at 25 and 37° C.

FIG. 9A presents representative QI mode AFM images of NanoART631 loaded with 2, 5, 7.5, 10, 12.5 and 15 wt % ART631 in water at 37° C., as indicated. FIG. 9B shows the average particle size of 100 particles of each NanoART631, calculated using ImageJ™. The particle sizes of the nanogels increase with increasing the ART631 loading content from 0 to 15 wt %.

FIG. 10 presents hydrodynamic diameter, polydispersity index (PDI), zeta potential and derived count rate of nanogels containing 0, 2, 5, 7.5, 10, 12.5 and 15 wt % ART631 in RPMI-1640 cell culture medium at 25 and 37° C.

FIG. 11A and FIG. 11B present in vitro cumulative release and daily release dose, respectively, of ART631 from nanogels in PBS (pH 7.4) as a function of time at 37° C. NanoART631 containing 2, 5 10, and 15 wt % ART631 sustained the release of ART631 at 37° C. for 35 days. The amount of ART631 released was quantified using peaks at 0.5-0.88 ppm obtained from 1H NMR CP800 MHZ, n=4.

FIG. 12 presents data on the cytotoxicity of plain nanogels and NanoART631 containing 2, 5 and 10 wt % ART631 to MOLM14 cells as a function of concentration in the range of 5e⁻⁶ and 10 mg/mL after 24 hours of incubation using MTT assay.

FIG. 13 presents data on the cytotoxicity of NanoART631 containing 5 and 10 wt % ART631 to MOLM14 cells as a function of concentration in the range of 0.0005 and 0.005 mg/mL after 24 hours of incubation using an MTT assay.

FIG. 14 presents data on the cytotoxicity of NanoART631 containing 2 wt % ART631 to MOLM14 cells as a function of concentration in the range of 0.005 and 0.01 mg/mL after 24 hours of incubation using an MTT assay.

FIG. 15 presents data for the IC₅₀ of NanoART631 loaded with 2, 5 or 10 wt % ART631 to MOLM14 cells after 24 hours of incubation using an MTT assay.

FIG. 16 presents data on the cytotoxicity of plain nanogels and NanoART631 containing 2, 5 and 10 wt % ART631 in a Normal Cell Model, adult retinal pigment epithelial (ARPE-19) cells as a function of concentration in the range of 0.005 and 1 mg/mL after 24 hours of incubation using an MTT assay.

FIG. 17 presents data on the cytotoxicity of plain nanogels and NanoART631 containing 2, 5 and 10 wt % ART631 in a Normal Cell Model, dental pulp stem cells (DPSCs) as a function of concentration in the range of 0.005 and 1 mg/mL after 24 hours of incubation using an MTT assay.

FIG. 18 presents data on the cytotoxicity of NanoART631 containing 5 wt % ART631 to MOLM14 cells as a function of concentration in the range of 5e⁻⁵ and 5e⁻³ mg/mL daily for 5 days using an MTT assay.

FIG. 19 presents data on the cytotoxicity of NanoART631 containing 5 wt % ART631 in a Normal Cell Model, adult retinal pigment epithelial (ARPE-19) cells as a function of concentration in the range of 5e⁻⁵ and 5e⁻³ mg/mL daily for 5 days using an MTT assay.

FIG. 20 presents data on the cytotoxicity of NanoART631 containing 5 wt % ART631 in a Normal Cell Model, dental pulp stem cells (DPSCs) as a function of concentration in the range of 5e⁻⁵ and 5e⁻³ mg/mL daily for 5 days using an MTT assay.

FIG. 21 presents data on the cytotoxicity of NanoART631 containing 5 wt % ART631 to MOLM14 cells, ARPE-19 cells and DPSCs at concentration 0.05 μg/mL as a function of time for 5 days using an MTT assay.

FIG. 22 presents data on the comparison of the effectiveness of ART631 and NanoART631 containing 5 wt % ART631 in killing MOLM14 Cells as a function of concentration in the range of 0.005 μg/mL and 1 mg/mL after 24 hours of incubation using an MTT assay.

FIG. 23A, FIG. 23B, and FIG. 23C present cytotoxicity of NanoART631 containing 2, 5 10, and 15 wt % ART631 to MOLM14, MV4;11 and THP1 cells against concentrations assessed by WST1 assay after 48-hour incubation at a 20,000 cells/well seeding density in 96-well plates (n=4), as indicated.

FIG. 24 presents data for the IC₅₀ of NanoART631 loaded with 2, 5, 10 or 15 wt % ART631 to MOLM14, MV4;11 and THP1 cells obtained by WST1 assay.

FIG. 25 presents the weekly IV dose of nanogels loaded with and without 5 wt % ART631 injected in nonobese diabetic/severe combined immunodeficiency gamma (NSG) female mice (n=3) for maximum tolerated doses (MTD) determination.

### DETAILED DESCRIPTION OF EMBODIMENTS OF THE INVENTION

### 1. Overview

This invention provides a polymerized nanogel that contains one or more drug compounds (preferably hydrophobic compounds, and most preferably hydrophobic compounds that contain one or more hydrophobic bonds) in aqueous solution. This nanogel pharmaceutical composition can be used for clinical use, including but not limited to cancer treatment and therapy (i.e., leukemic, skin, melanoma, lung, bronchus, kidney, liver, breast, oral, head, neck, esophageal, thyroid, eye, retinal, ear, bone, cartilage, fat, muscle, blood vessel, gastrointestinal stromal, intrahepatic bile duct, bladder, colon, rectum, vaginal, prostate, testicular, pancreatic, cervical, uterine, pleural, immune system, glioblastoma, non-Hodgkin lymphoma, carcinoma, adenocarcinoma, basal cell carcinoma, squamous cell carcinoma and transitional cell carcinoma, sarcoma, lymphoma, myeloma, brain and spinal cord cancers), and the like, or any disease or condition that would benefit from long-term sustained release of an active agent for treatment). For example, diseases and conditions such as malarial, fungal, infection, inflammation, seizure, stroke, depression, hepatitis C, diabetes, diabetic retinopathy, age-related macular degeneration, glaucoma, dry eye, Alzheimer's disease, Parkinson's disease, neurological disorders, pain, temporomandibular joint disorders, and opioid overdose can by treated using the nanogel delivery system of the invention. The diseases and conditions may occur in any tissue and organ including but not limited to connective, epithelial, muscle or nervous tissue; and bone, cartilage, tendon, ligament, joint, tooth, nerve, blood vessel, artery, vein, capillary, lymphatic vessel, muscle, skin, heart, brain, skull, hypothalamus, cerebellum, kidney, liver, lung, ear, eye, cornea, lens, retina, vitreous, optic nerve, nose, olfactory epithelium, face, mouth, tongue, salivary gland, larynx, thymus gland, thyroid, trachea pancreas, spinal cord, stomach, small intestine, large intestine, cecum, colon, rectum, anus, genital, bladder, spleen, ureter, urethra, uterus, vagina, penis, scrotum, prostate, hair, testes or nail.

Using this nanogel, and preferably by copolymerizing a hydrophobic drug to the nanogel components, increases water solubility, half-life, and consequently bioavailability of the drug, and provides improved drug efficacy. In a preferred embodiment, the invention involves copolymerizing the hydrophobic compound with N-isopropylacrylamide monomer and dextran-lactate⁻²-hydroxyethyl-methacrylate macromer via UV emulsion polymerization in aqueous solution. The resulting pharmaceutical system has the additional advantage of being able to sustain the release of the drug candidate for a long period of time, thus increasing therapeutic effects of the compounds, reducing the need for frequent dosing, and increasing patient compliance.

Specifically, a preferred embodiment of the invention is a nanogel system that can significantly increase the water solubility of drugs such as ART631 by copolymerizing it into the nanogels using a UV emulsion polymerization technique. The obtained composition (referred to here as NanoART631) maintains ART631 activity against human acute myeloid leukemia MOLM14 cells. Both the nanogels alone and NanoART631 were not toxic to two nonmalignant cell lines: adult retinal pigment epithelium-19 (ARPE-19) cells and dental pulp stem cells (DPSCs) and were well tolerated by NRG mice at a dose of 500 mg/kg IV. Thus, the invention, including embodiments such as NanoART631 has potential as a novel active pharmaceutical delivery agent that addresses the limited solubility and in vivo half-life of certain drugs. In certain embodiments, the 5 wt % ART631-containing nanogels can increase the water solubility of ART631 more than 400 fold. The nanogels can be loaded with about 15 wt % ART631 into the nanogels or more, including up to about 30 wt % ART631 and also up to about 80 wt %. Preferably, the gels are loaded with about 1 to about 50 wt %, and most preferably about 1 to about 30 wt % of the drug composition. At a loading of 80 wt %, the water solubility of ART631 can be increased to 400×(80 wt %/5 wt %)=6400 fold.

### 2. Definitions

Unless defined otherwise, all technical and scientific terms used herein have the same meaning as commonly understood by one of ordinary skill in the art. Although various methods and materials similar or equivalent to those described herein can be used in the practice or testing of the present invention, suitable methods and materials are described below. However, the skilled artisan understands that the methods and materials used and described are examples and may not be the only ones suitable for use in the invention. Moreover, as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

As used herein, the term “about” means plus or minus 20 percent of the recited value, so that, for example, “about 0.125” means 0.125±0.025, and “about 1.0” means 1.0±0.2.

Notwithstanding that the numerical ranges and parameters setting forth the broad scope of the invention are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Moreover, all ranges disclosed herein are to be understood to encompass any and all sub-ranges subsumed therein. For example, a range of “less than 10” includes any and all sub-ranges between (and including) the minimum value of zero and the maximum value of 10, that is, any and all sub-ranges having a minimum value of equal to or greater than zero and a maximum value of equal to or less than 10, e.g., 1 to 4.

As used herein, the term “cancer” refers to any hyperproliferative disorder that is treatable using a chemotherapeutic drug. The invention described herein is contemplated for use with any of such cancers, including but not limited to, leukemias, skin, melanoma, lung, bronchus, kidney, liver, breast, oral, head, neck, esophageal, thyroid, eye, retinal, ear, bone, cartilage, fat, muscle, blood vessel, gastrointestinal stromal, intrahepatic bile duct, bladder, colon, rectum, vaginal, prostate, testicular, pancreatic, cervical, uterine, pleural, immune system, glioblastoma, non-Hodgkin lymphoma, carcinoma, adenocarcinoma, basal cell carcinoma, squamous cell carcinoma and transitional cell carcinoma, sarcoma, lymphoma, myeloma, brain and spinal cord cancers, and the like, or any disease or condition that would benefit from long-term sustained release of an active agent for treatment.

As used herein, the term “leukemia” refers to several different cancers of the blood and the blood-forming cells of the body. Most often, leukemia is a cancer of the white blood cells, but some leukemias start in other blood cell types. There are several types of leukemia, which are divided based mainly on whether the leukemia is acute (fast growing) or chronic (slower growing), and whether it starts in myeloid cells or lymphoid cells. Different types of leukemia have different treatment options and outlooks. Types of leukemia comprises acute lymphocytic leukemia (ALL), acute myeloid leukemia (AML), chronic lymphocytic leukemia (CLL). chronic myeloid leukemia (CML), B-Cell prolymphocytic leukemia (B-PLL). blastic plasmacytoid dendritic cell neoplasm (BPDCN), chronic myelomonocytic leukemia (CMML), hairy cell leukemia, juvenile myelomonocytic leukemia (JMML), large granular lymphocytic leukemia (LGLL), and T-cell prolymphocytic leukemia (T-PLL).

As used herein, the terms “subject,” “individual,” “host,” and “patient,” are used interchangeably to refer to any animal, and can include humans, simians, avians, felines, canines, equines, rodents, bovines, porcines, ovines, caprines, mammalian farm animals, mammalian sport animals, and mammalian pets. A “subject in need” refers to a subject suffering from or likely to be suffering from a disease or condition that can be benefitted by administration of a drug in a sustained release injectable form as described herein, for example an artemisinin or its derivatives, osimertinib, sunitinib, quinine, lumefantrine, stiripentol, glecaprevir, cyclosporin, voclosporin, naloxone, betulinic acid, bevirimat, derivatives, dienestrol, neuroprotection, prostaglandin, unsaturated fatty acids, rilpivirine, polyene antimycotics, or a combination thereof.

As used herein, the term “initiator” refers to a source of any chemical species that reacts with a monomer to form an intermediate compound capable of linking successively with a large number of other monomers to form a polymeric compound. In the context of this invention, the initiator preferably is selected from the group consisting of 2-hydroxy-4′-(2-hydroxyethoxy)-2-methylpropiophenone, 2,2′-azobis [2-(2-imidazolin-2-yl) propane] dihydrochloride, 1-hydroxycyclohexylphenyl-ketone, 2-hydroxy-2-methyl-1-phenylpropanone, 2,2-dimethoxy-1,2-diphenyl-ethan-1-one, 2-(4-Methylbenzyl)-2-(dimethylamino)-1-(4-morpholinophenyl) butan-1-one, alpha hydroxy ketones, phosphine oxides, benzophenone, thioxanthones, 2,2-dimethoxy-2-phenylacetophenone, isopropyl thioxanthone, 2-ethylhexyl-(4-N,N-dimethyl amino)benzoate, ethyl-4-(dimethylamino)benzoate, peroxides, benzoyl peroxide, molecular oxygen, azobisisobutyronitrile, camphorquinone, eosin Y, triethanolamine, 1-vinyl-2-pyrrolidinone, or a combination thereof.

As used herein, the term “macromer” refers to structures according to Formula I:

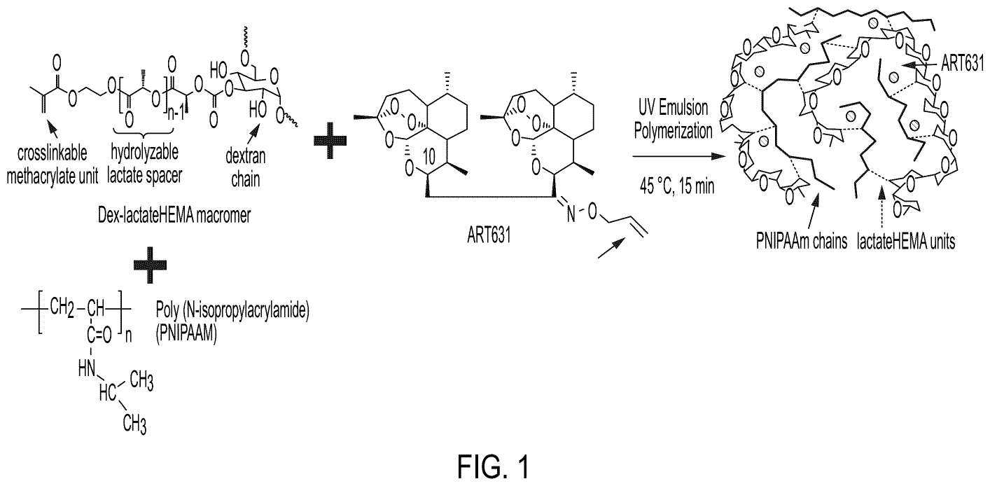

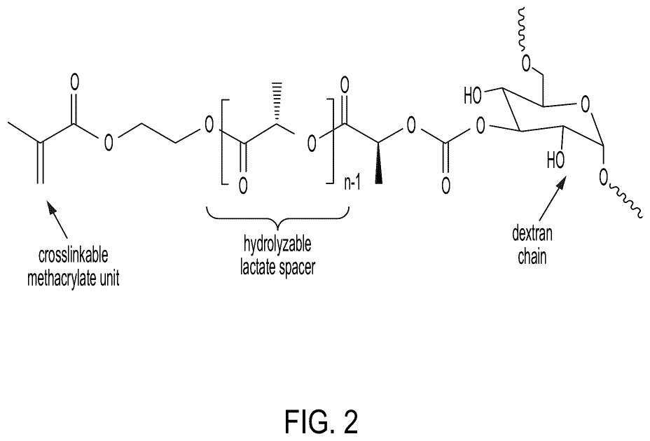

CLU-HS-DU,

where CLU is a crosslinkable unit, HS is a hydrolysable spacer, and DU is a dextran unit. Preferably, the macromer is an oligolactate-(2-hydroxyethyl methacrylate) (PLA-HEMA) grafted dextran molecules (Dex-PLA-HEMA) or a poly-ε-caprolactone-(2-hydroxyethyl methacrylate (Dex-PCL-HEMA) grafted dextran molecules used in the production of the nanogels. For an example, see FIG. 1 and FIG. 2.

In certain preferred embodiments, the DEX-PLA-HEMA macromer has polylactic acid component which is hydrolytically and enzymatically degradable. When a macromer or monomer has two or more double bonds, a crosslinked structure can be obtained, which is biodegradable. A preferred macromer is hydrolysable and contains two or more double bonds in order to provide this feature.

Appropriate macromers for use with the invention include hydrolyzable molecules composed of dextran, polylactic acid, polylactic-co-glycolic acid, polyethylene glycol, poly(ethylene oxide)-poly(propylene oxide)-poly(ethylene oxide), poly(ethylene oxide)-co-poly(L-lactic acid), biotinylated poly(ethylene glycol-block-lactic acid), pluronic acid, polaxamer, polyesters, polyamides, poly(amino acid), polyurethane, polyorthoesters, polyanhydrides, polyethylene terephthalate, polycarbonates, polyfumarates, polycyanoacrylates, poly(alkylcyanoacrylate), polyphosphazenes, polyphosphoesters, or poly(bis(p-carboxyphenoxy) propane-sebacic acid), or a combination thereof, wherein the hydrolysable molecule contains one or more double bonds.

As used herein, the term “monomer” refers to an N-isopropylacrylamide (NiPAAm), N-alkylacrylamide, N-n-propylacrylamide, N-isopropylmethacrylamide, or any combination thereof, and preferably is N-isopropylacrylamide.

As used herein, the term “degree of polymerization (DP)” refers to the average number of units on a polymer molecule, for example the number of lactide units on the PLA-HEMA segment of a macromer.

As used herein, the term “degree of substitution (DS) refers to the average number of units per length of macromer, for example the number of PLA-HEMA units per 100 glucose units of dextran.

As used herein, the term “nanoparticle” refers to particles that have a size of about 1 nm to 1000 nm.

As used herein, the term “nanogel” refers to crosslinked particles that have a size of about 1 nm to 1000 nm, preferably about 1 to 600 nm, and most preferably about 10 to about 350 nm.

### 3. Embodiments of the Invention

### A. Introduction

The general strategy is as follows. The 2-C ART Dimers are designed to improve bioavailability and half-life, and are more potent (IC50<50 nM) than artesunate (AS) based on 9 human leukemia cell lines in vitro. See U.S. Pat. No. 9,487,538, incorporated by reference herein. ART631, although water insoluble, is among the most potent analogs (IC50<45 nM) with improved oral bioavailability. This application describes a biodegradable nanogel system that can significantly increase the water solubility of hydrophobic drugs, for example 2C-ARTs, ART631, and preferably such drugs containing double bonds, by enveloping the drug in a polymerized nanogels using UV emulsion polymerization technique. In certain embodiments, the nanogel compositions according to this invention are N-isopropylacrylamide monomer and dextran-lactate⁻²-hydroxyethyl-methacrylate macromer crosslinked via UV irradiation in aqueous solution.

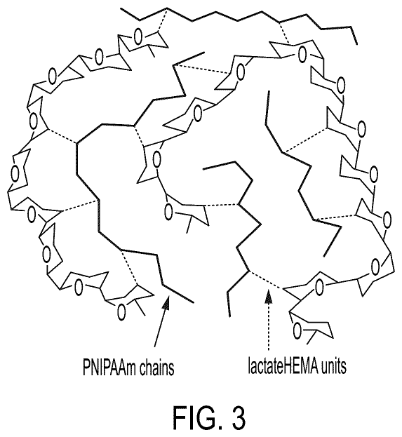

A UV emulsion polymerization technique was used to polymerize ART631 through its double bond to the dextran-polylactic acid (DEX-PLA-HEMA) poly(N-isopropylacrylamamide) (pNIPAAM) nanogels. The emulsion polymerization time for preferred nanogel compositions was about 15 minutes. However, for some other embodiments, the reaction time can be as low as about 2 minutes and up to about 5 hours to form/obtain the nanogels. The aim is to increase the solubility of ART631 in water and to sustain its release. See FIG. 2 and FIG. 3 for the chemical structure and drawing of the dextran-polylactic acid (DEX-PLA-HEMA) poly(N-isopropylacrylamamide) (pNIPAAM) nanogel.

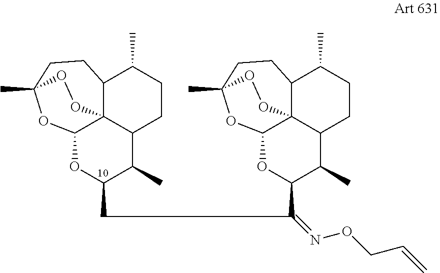

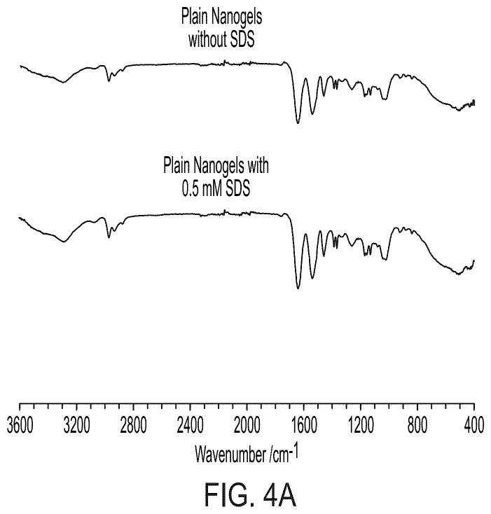

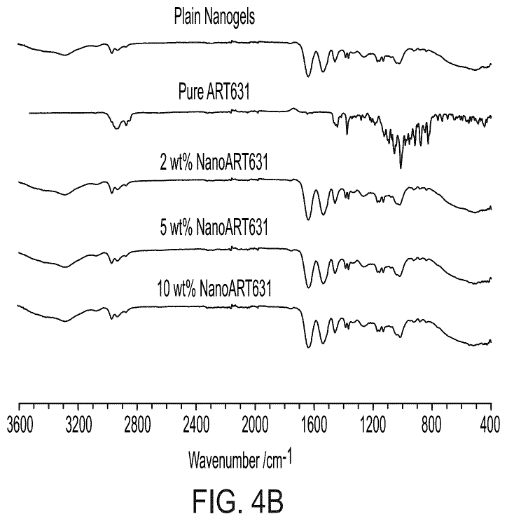

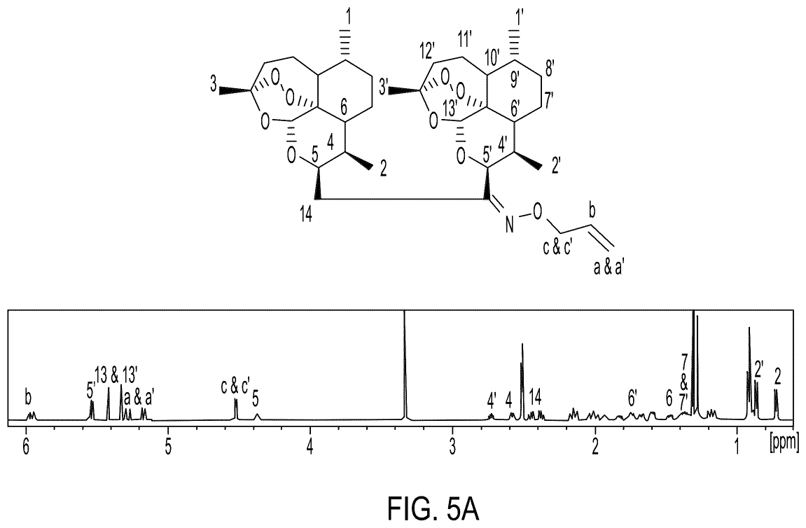

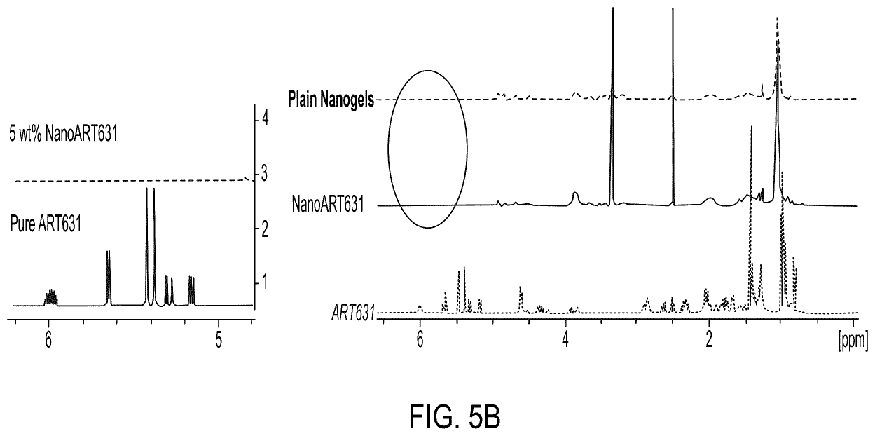

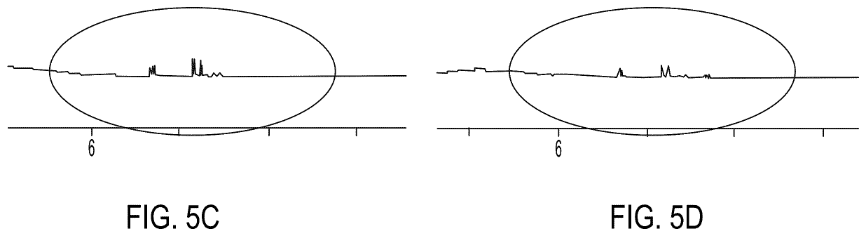

The results in FIG. 4 and FIG. 5 demonstrate that nanogels can provide sustained release of ART838 into PBS over days-weeks. This delayed release profile might be beneficial to potentiate antitumor responses initiated by other drug agents. Therefore, it is contemplated

Results presented here have demonstrate that 2C-ART dimers are potent antileukemic agents compared to artesunate. ART631 loading to nanogel formulations according to embodiments of the invention improved its water solubility >400-folds from lower than 10 μg/mL to up to at least 4 mg/mL and also will sustain its release for at least 35 days. In addition, NanoART631 has an IC50 of about 50 nM or lower after a one-day incubation with human AML MOLM 14 cells and killed 88% and 98% of the AML cells at 4 nM after 2 and 3 days of incubation, respectively. Nanogels with or without ART631 were not toxic to two nonmalignant cell lines and were well tolerated by NRG mice at a dose of 500 mg/kg IV (25 mg/kg of ART631).

### B. Loading of Nanogel Formulations

To load the nanogel, the ART631 or another drug to be loaded is included in the synthetic mixture. Generally, a concentration of about 1 wt % to about 80 wt %, preferably about 1 wt % to about 50 wt %, and most preferably about 1 wt % to about 30 wt % of the drug can be used. The amount can easily be determined by the practitioner depending on the nature of the biologic(s), the dose required for a particular patient and disease or condition from which the patient suffers. Quantitation of the loaded medicament in the nanogel can be determined using an ultra performance liquid chromatography (UPLC) or UPLC/MS-MS method. See Examples, below.

The percentage yield is calculated as ((theoretical nanogel weight−the amount of nanogel recovered)/theoretical weight of the nanogel×100). Percent ART631 loading is calculated as (total amount of ART631 loaded measured by ¹H NMR/total amount of ART631 loaded×100).

### C. Characterization of the Nanogel System

The nanogels increased ART631 water solubility >400-folds from lower than 10 μg/mL to up to at least 4 mg/mL. The z-average diameter of the ART631-loaded nanogels (NanoART631) was 100-200 nm in DI water at 37° C., with a polydispersity index <0.3 and a −11-−15 mV zeta potential.

### D. Cytotoxicity and IC₅₀

The NanoART631 effectively killed MOLM 14, MV4;11 and THP1 cells with IC50 in the nanomole concentration using 3-(4,5-dimethylthiazol-2-yl)-2,5-diphenyl-2H-tetrazolium bromide (MTT) and WTS1 assays. In contrast, NanoART631 was not toxic to two nonmalignant adult retinal pigment epithelium-19 (ARPE-19) cells and dental pulp stem cells (DPSCs) at a nanoparticle concentration of 1 mg·mL-1, which contained ˜160 μM ART631.

### E. In Vivo Toxicology

The single-dose IV maximal tolerated dose (MTD) of NanoART631 in highly immunodeficient NRG mice (n=3) was 500 mg/kg (containing ˜25 mg/kg ART631). Empty nanoparticles (without ART631) had no clinical toxicity to mice at the same and higher IV dose up to at least 800 mg/kg.

### F. Diseases and Conditions

Any disease or condition that can benefit from longer term administration of a drug, particularly a hydrophobic drug such as artemisinin or its derivatives (including ART631) or is suitable for treatment using the invention described herein is contemplated for treatment using the invention. Such conditions include, cancer, in particular leukemia, and in more particular acute myeloid leukemia. The diseases and conditions can also be skin, melanoma, lung, bronchus, kidney, liver, breast, oral, head, neck, esophageal, thyroid, eye, retinal, ear, bone, cartilage, fat, muscle, blood vessel, gastrointestinal stromal, intrahepatic bile duct, bladder, colon, rectum, vaginal, prostate, testicular, pancreatic, cervical, uterine, pleural, immune system, glioblastoma, Non-Hodgkin lymphoma, carcinoma-adenocarcinoma, basal cell carcinoma, squamous cell carcinoma and transitional cell carcinoma, sarcoma, lymphoma, myeloma, brain and spinal cord cancers, and the like, or any disease or condition that would benefit from long-term sustained release of an active agent for treatment. For example, diseases and conditions such as malarial, fungal, infection, inflammation, seizure, stroke, depression, hepatitis C, diabetes, diabetic retinopathy, age-related macular degeneration, glaucoma, dry eye, Alzheimer's disease, Parkinson's disease, neurological disorders, pain, temporomandibular joint disorders, and opioid overdose can by treated using the nanogel delivery system of the invention.

The invention can be used in treating any tissue or organ of the body, including but not limited to connective, epithelial, muscle; and bone, cartilage, tendon, ligament, joint, tooth, nerve, blood vessel, artery, vein, capillary, lymphatic vessel, muscle, skin, heart, brain, skull, hypothalamus, cerebellum, kidney, liver, lung, ear, eye, cornea, lens, retina, vitreous, optic nerve, nose, olfactory epithelium, face, mouth, tongue, salivary gland, larynx, thymus gland, thyroid, trachea pancreas, spinal cord, stomach, small intestine, large intestine, cecum, colon, rectum, anus, genital, bladder, spleen, ureter, urethra, uterus, vagina, penis, scrotum, prostate, hair, testes or nail, and the like.

### G. Methods of Use

The nanogels are administered to a patient in a location such that the nanogel and the drug released from the nanogel will be released in or near the affected tissue, such as a tumor. Preferably, a solution containing the nanogels is injected into the appropriate tissue. The solution used and the injection volume will depend on the concentration of nanogels and the location of the injection. The practitioner is able to determine an appropriate route of administration and amount.

Nanogels preferably are prepared as a suspension in a solution of water, PBS, medium, or the like, as determined by the practitioner, at a concentration of about 1e⁻⁶ to about 100 mg/mL, preferably about 1e⁻⁴ to about 10 mg/mL, and most preferably about 1⁻³ to about 10 mg/mL. A dose of the nanogel suspension generally is given at about 1 μL to about 10 mL, and more preferably about 1 μL to about 2 mL, depending on the tissue or area of the body to be injected. Thus, an individual dose generally is about 1 μg/kg to about 2 g/kg. Doses may be administered periodically to maintain drug release for an appropriate time as determined by the clinician. For example, administration of nanogel formulations of drug can be administered weekly, biweekly, every three weeks, monthly, every two months, quarterly, semi-annually, or any frequency. Any dosage schedule is appropriate depending on the release rate, total amount of ART631 in the administered nanogels, and the condition of the patient.

Any tissue or organ in need of treatment by ART631 can be treated according to the invention. For example, ART631 loaded onto nanogels can be administered by injections, such as intravenous, intramuscular, subcutaneous, intradermal, transdermal, intraperitoneal, parenteral, intracarotid, intraarterial, intratumoral, or other routes. Other examples include, but are not limited to, intrathecal, intracerebroventricular, intraparenchymal, intracranial, intravesicular, intraocular, intravitreal, subconjunctival, subretinal, intrathyroidal, intrathoracic, intrachoroidal, oral/gastro-intestinal, buccal/sublingual, topical, intra-tympanic, intramucosal, intrarectal, vaginal, intraurethral, uterine, intracervical, intranasal, inhalation, or intraosseous.

### 5. Examples

This invention is not limited to the particular processes, compositions, or methodologies described, as these may vary. The terminology used in the description is for the purpose of describing the particular versions or embodiments only, and is not intended to limit the scope of the present invention which will be limited only by the appended claims. Although any methods and materials similar or equivalent to those described herein can be used in the practice or testing of embodiments of the present invention, the preferred methods, devices, and materials are now described. All publications mentioned herein, are incorporated by reference in their entirety; nothing herein is to be construed as an admission that the invention is not entitled to antedate such disclosure by virtue of prior invention.

### Example. 1. Production and Testing of Nanogel/Nanoparticle Delivery System

Nanogels composed of poly(N-isopropylacrylamide) and dextran-poly(lactate⁻²-hydroxyethyl-methacrylate) were used to load ART631. Nanoparticle size was measured by dynamic light scattering (DLS) and atomic force microscopy (AFM). Zeta potential was measured by Zetasizer. In vitro cytotoxicity of nanogels, with and without AR631, against MOLM14 cells, ARPE-19 cells and DPSCs was evaluated via 3-(4,5-dimethylthiazol-2-yl)-2,5-diphenyl-2H-tetrazolium bromide (MTT) or WST1 assays over 5 days. In vivo toxicology of the nanogel-drug system was evaluated by intravenous (IV) injection of the nanogel-drug system into highly immunodeficient NRG mice (n=3).

Results were as follows. The nanogels increased ART631 water solubility >400-fold from lower than 10 μg/mL to up to at least 4 mg/mL. ART631-loaded nanogels (NanoART631) were monodisperse and their z-average diameters were 100-200 nm with polydispersity index <0.3 and a −9-−16 mV zeta potential in DI water at 37° C., and 100-260 nm with a polydispersity index <0.35 and a −7-−11 mV zeta potential in RPMI-1640 cell culture medium at 37° C. The particle diameters measured by AFM were 20-50 nm in wet state at 37° C. In MTT assays, NanoART631 formulation had IC50 ˜50 nM (based on estimated ART631 composition) after one day incubation with MOLM 14 cells and killed 88% and 98% of the AML cells at 4 nM after 2 and 3 days of incubation, respectively (similar to the antileukemic activity of “soluble” ART631). In contrast, NanoART631 was not toxic to ARPE-19 cells or DPSCs at a nanoparticle concentration of 1 mg/mL, which contained about 160 μM ART631. In the WST1 assay, NanoART631 was very potent in killing all the three cell lines MOLM14, MV4;11 and THP1 cells after 48 hours incubation at a 20,000 cells/well seeding density in 96-well plates with IC50 concentration lower than 35 nM. The single-dose IV maximal tolerated dose (MTD) of NanoART631 in NRG mice (n=3) was 500 mg/kg (containing about 25 mg/kg ART631). Empty nanoparticles (without ART631) had no clinical toxicity to mice at the same and higher IV dose up to at least 800 mg/kg.

NanoART631 can be used as an active antileukemic agent that addresses the limited solubility and in vivo half-life of artemisinin analogs.

### Example 2: Synthesis of Plain (Unloaded) and ART631-Loaded Nanogels

Nanogels containing 0, 2, 5, 7.5, 10, 12.5 and 15 wt % ART631 were synthesized by UV emulsion polymerization in an ethanol/water aqueous solution containing 0.5 mM SDS (see Scheme 1). Plain (unloaded) nanogels synthesized with and without 0.5 mM SDS were used as controls.

Additionally, plain biodegradable nanogels containing N-isopropylacrylamide (NIPAAm) and dextran grafted oligolactate-(2-hydroxyetheyl methacrylate) (Dex-PLA-HEMA, DP=6 and DS=8.7) macromer at weight ratio 7:2 (NIPAAM: macromer) were synthesized by UV emulsion polymerization using Irgacure® 2959 (2-hydroxy-4′-(2-hydroxyethyl)-2-methyl propiophenone), as an initiator. During synthesis, the monomer and macromer were dissolved in DI water (10 mg/mL), and degassed for 10 minutes using N₂ gas in an oil bath (45° C.). While still purging with nitrogen at 45° C., the solution was exposed to UV light (1 W/cm²) at a distance approximately 1.5 inches from an EXFO Lite UV curing light source (EXFO, Inc.) under stirring at 300 rpm and 45° C. for 15 minutes at 320-500 nm wavelength.

Optionally the nanogel contains sodium dodecyl sulfate (SDS), in which case 0.5 mM SDS was added to the water for the synthesis described above.

ART631-loaded nanogels (NanoART631) containing 2, 5, 7.5, 10, 12.5, or 15 wt % of the combined weight of NIMPAAM and DEX-PLA-HEMA were synthesized by dissolving ART631 in ethanol first before adding the ethanol solution to the reaction mixture with SDS for UV emulsion polymerization. Scheme 1, shown in FIG. 1, illustrates the nanogel synthesis process.

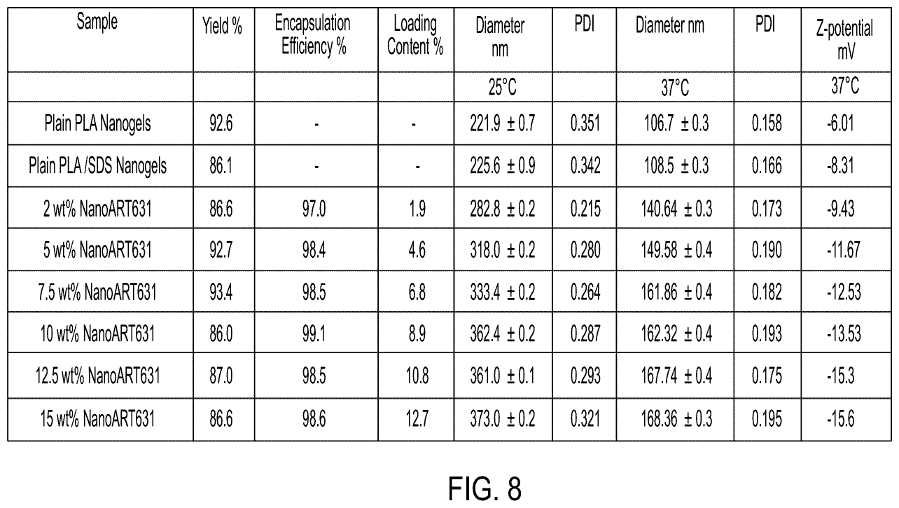

After the synthesis, the nanogels were dialyzed in 5 L DI water through a dialysis membrane (Spectra/Por® 6 Dialysis Membrane MWCO: 50 kD, Spectrum Laboratories Inc.) for 5 hours, with change of the dialysis media every 30 minutes. The purified nanogel dispersion was lyophilized to obtain dry nanogels. FIG. 8 shows that all the nanogels were synthesized with a yield more than 86%. The nanogels increased ART631 water solubility >400-fold from lower than 10 μg/mL to up to at least 4 mg/mL.

### Example 3. Chemical Structure of Synthesized Nanogels

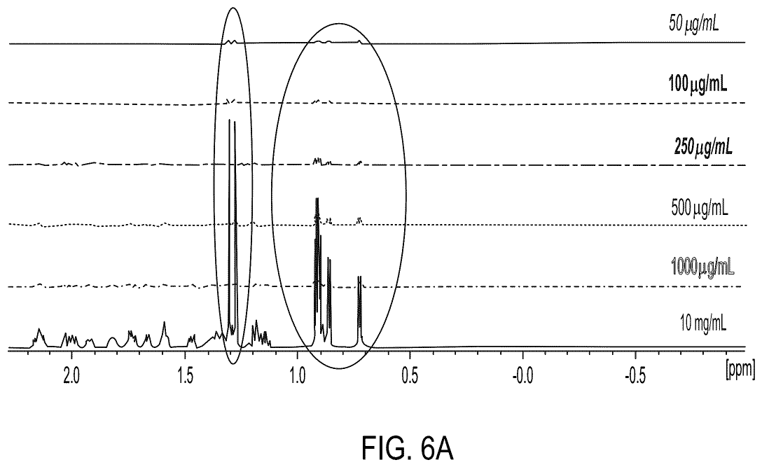

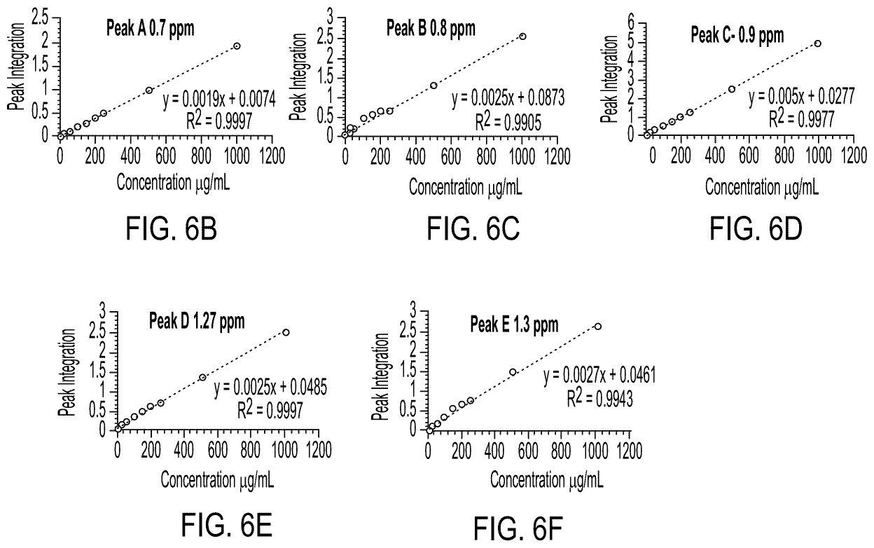

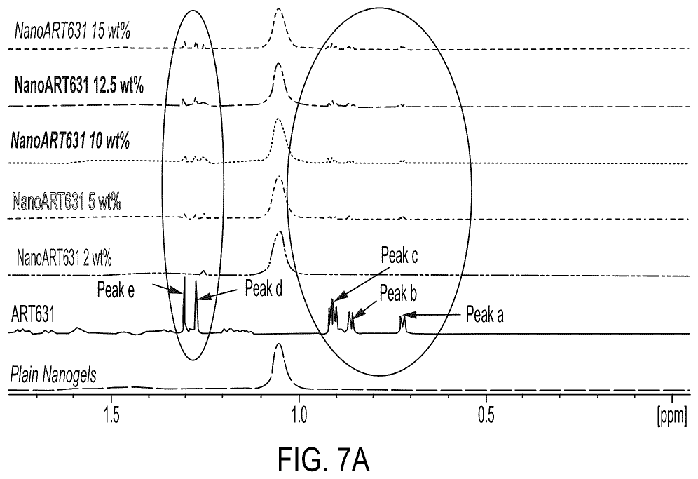

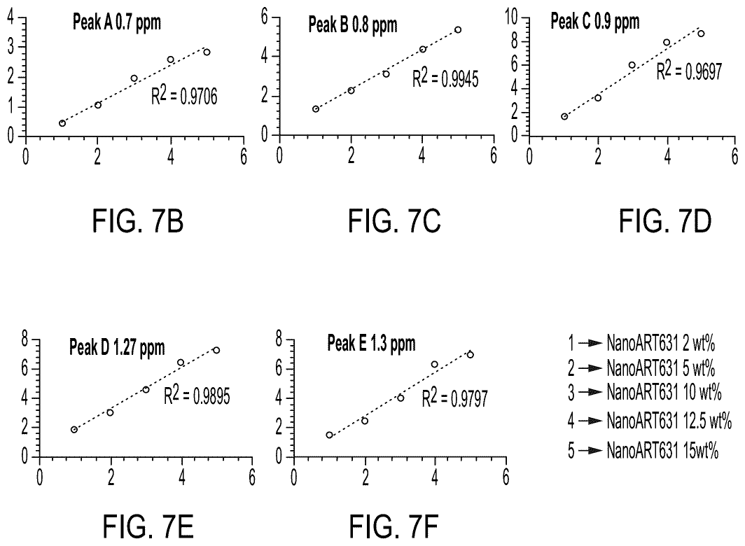

The chemical structures of the nanogels, empty and loaded with ART631, were characterized by Fourier transform infrared spectra (FTIR) and 1H nuclear magnetic resonance (NMR). See FIG. 4, FIG. 5, FIG. 6 and FIG. 7. FTIR spectra were obtained by using Nicolet 6700 FTIR spectrometer (Thermo Electron Corporation, Wilmington, DE, USA). ¹H NMR spectra were obtained by using Bruker AV III Ultrashield Plus™ 600 MHZ with a TXI 600SB H-C/N-D-05 Z BTO probe, Bruker AV III Ascend CP800 MHz with a CPQCI 1H-31P/13C/15N/D Z-GRD cryoprobe, and Bruker AV III 950 MHz with a TCI cryogenic probe NMR spectrometers (Brucker™). All ART631 and NanoART631 were dispersed in deuterated dimethyl sulfoxide (DMSO) at room temperature.

Plain nanogels and 0.5 mM SDS plain nanogels were synthesized as described above, and their synthesis was confirmed by NMR and FTIR (see FIG. 4A). For these tests, to load ART631, the drug was first dissolved in ethanol and then polymerized with the C═C bond of the DEX-PLA-HEMA. This polymerization was first confirmed by the opaque physical appearance of the formulation, compared to the clear appearance of the plain nanogels and 0.5 mM SDS plain nanogels. Secondly, 1H NMR was used to observe the ART631 C═C peak disappearance (see FIG. 5B). These peaks were identified as hydrogens p (δ≈6.0 ppm), q and r at δ≈5.16 and 5.28 ppm. Additionally, hydrogen h at ≈≈5.4 ppm and hydrogen I at δ≈5.65 ppm can also be affected and become more shielded due to the polymerization process.

In FIG. 4A, the presence of the characteristic peaks: C═O stretching of ester of DEX-PLA-HEMA at 1750 cm⁻¹, C—O stretching of amide I and N—H bending and C—N stretching of amide II of PNIPAAM at 1650 and 1540 cm⁻¹, respectively, coupling between symmetric bending of the two neighboring methyl groups of the isopropyl group-CH(CH₃)₂ of PNIPAAM at 1386 and 1369 cm⁻¹, and C—OH stretching of dextran at 1020 cm⁻¹, confirm the successful synthesis of the plain nanogels. The similarity of the plain nanogels with and without 0.5 mM SDS suggests that the addition of 0.5 mM SDS has no effect on the FTIR spectrum of the nanogels.

FIG. 4B shows that after ART631 is polymerized into the nanogels, the intensity of peak at 1020 cm⁻¹ increases with increasing the amount of ART631 from 0, 2, 5 to 10 wt %, due to the contribution of the substituted C—H bending of ART631.

The ¹H NMR spectrum in FIG. 5A shows that ART631 has peaks at chemical shift δ≈6.0, 5.16, 5.28, and 5.4 ppm, corresponding to its C═C double bond. After ART631 was copolymerized into the nanogels after UV emulsion polymerization, the four peaks disappear. The characteristic peaks of ART631 at 0.7, 0.8, 0.9, 1.27, and 1.3 ppm regions appear in NanoART631 (see FIG. 5B). When ART631 is physically mixed with plain PLA/SDS nanogels in both full amount (see FIG. 5C) and only half amount (see FIG. 5D) of ART631, the characteristic peaks of ART631 reappear. These findings eliminate the possibility that the peaks disappeared due to the high plain nanogel: ART631 ratio. Taken together, the data discussed here demonstrate that ART631 is successfully co-polymerized into the nanogels during the UV emulsion polymerization process.

### Example 4. ART631 Encapsulation Efficiency and Loading Content

1D proton qNMR was used to measure ART631 and calculate its encapsulation efficiency (EE %) and loading content (LC %) percentages in NanoART631 by monitoring five ART631 peaks at the 0.7, 0.8, 0.9, 1.27, and 1.3 ppm regions. The intensities of the five peaks were normalized to an internal standard 3,4,5-trichloropyridine using the following equation to quantify ART631 amount:

$$\frac{{I}_{A}}{{I}_{B}} = \frac{{H}_{A} {C}_{A}}{{H}_{B} {C}_{B}}$$

I=Signal intensity (Integral), H=Number of protons in a functional group,
C═Concentration

Since ART631 is covalently bonded to the nanogels, when ART631 is released by nanogel degradation, the drug can be released with several different fragments of the nanogels attached. This scenario makes it difficult to measure ART631 with conventional HPLC, UPLC, or mass spectrometry techniques. Therefore, a new method for quantitation of ART631 using NMR is developed. FIG. 6A presents 1H NMR spectra of ART631 at different concentrations and shows that the intensities of the five ART631 peaks at chemical shift 0.7, 0.8, 0.9, 1.27, and 1.3 ppm regions increase with increasing ART631 concentration from 50 μg/mL to 10 mg/mL, which can be used as standard curves for determining the amount of ART631 as shown in FIG. 6B. By using Bruker AV III 950 MHz NMR, these peaks can be used to determine ART631 with high sensitivity down to 450 ng/ml concentration. No peaks at the 5-6 ppm region are present, while all other nanogel peaks are present.

Accordingly, NanoART631 loaded with 2, 5, 7.5, 10, 12.5 and 15 wt % ART631 were measured by ¹H NMR and the five characteristic peaks of ART631 at chemical shifts 0.7, 0.8, 0.9, 1.27, and 1.3 ppm regions were used to measure the amount of ART631 based on the standard curves in FIG. 6B, and to calculate the encapsulation efficiency (EE %) and loading content (LC %) using the following equations:

$$EE \% = \frac{Amount of loaded drug}{Total drug} \times 100 \\ Amount of loaded drug = Total drug - unloaded drug \\ LC ( \% ) = \frac{Mass of the loaded drug in the nano carrier}{Nano carrier mass} \times 100$$

FIG. 7B shows that the quantitated ART631 loading amount in the nanogels linearly increases with increasing the theoretical ART631 loading content from 2, 5, 10, 12.5 to 15 wt % at all five characteristic peaks of ART631. FIG. 8 demonstrates that ART631 is successfully loaded into the nanogels with all the range of ART631 theoretical loading content from 2 to 15 wt % during the synthesis with calculated EE % and LC % to be more than 97% and 1.9-12.7%, respectively.

### Example 5. Particle Size and Zeta Potential

The z-average hydrodynamic diameter, polydispersity index (PDI) and zeta potential of the freshly prepared plain and ART631-loaded nanogels in DI water and RPMI-1640 cell culture medium at 500 μg/mL were measured at 90° C. and 25 and 37° C. using ALV dynamic light scattering containing an ALV-CGS-8F compact goniometer system, a DPSS laser (660 nm, 50 mW), and an ALV-5000/EPP multiple tau digital real time correlator (ALV-Laser Vertriebsgesellschaft m.b.H., Langen, Germany). The zeta potential of the nanogels in DI water and RPMI-1640 medium at 500 μg/mL was measured at 25 and 37° C. using Zetasizer Nano ZS (Malvern™). The sample dispersions were filtered through 0.45 μm syringe filters before the measurements. The Stokes-Einstein equation was used to determine the particle diameter from the diffusion intensity, and NNLS analysis was used to determine the size distributions. Derived count rates were reported after taking the attenuation into consideration. Three replicates were performed for each measurement and reported as the mean±standard deviation.

Atomic Force Microscopy (AFM) with quantitative imaging mode (JPK NanoWizard 4a AFM QI Mode, Brucker™) was used to measure single particle size for plain and different wt % ART631 loaded nanogels by plating 10 μg/mL samples dissolved in water on mica substrate. Nanogels on mica were dried by nitrogen air, and then samples were rehydrated when mounted on the AFM and then equilibrated at 37° C. before being imaged. A C14 probe was used, a 2×2 μm map was imaged, and several AFM regions of interest (2-3 regions) were captured per nanogel formulation. A total of 100 particles were sized using ImageJ™ per formulation, and the quantities were reported as the mean±standard deviation.

FIG. 8 shows that the plain nanogels without SDS have a particle hydrodynamic diameter of 221.9±0.72 nm at 25° C. and the size becomes smaller, 106.7±0.29 nm at 37° C. due to the thermo-responsive property of PNIMAAM. Addition of 0.5 mM SDS during the synthesis increases the particle size slightly (225.6±0.86 vs. 221.9±0.72 nm at 25° C., and 108.5±0.34 nm vs. 106.7±0.29 nm at 37° C.) due to the negative charges of SDS. After ART631 is synthesized into the nanogels, the particle size increases with increasing the ART631 loading content from 0 to 15 wt % at both 25 and 37° C. The polydispersity index (PDI) values of all the nanogels are below 0.2 at 37° C., suggesting that the synthesized nanogels are quite monodisperse.

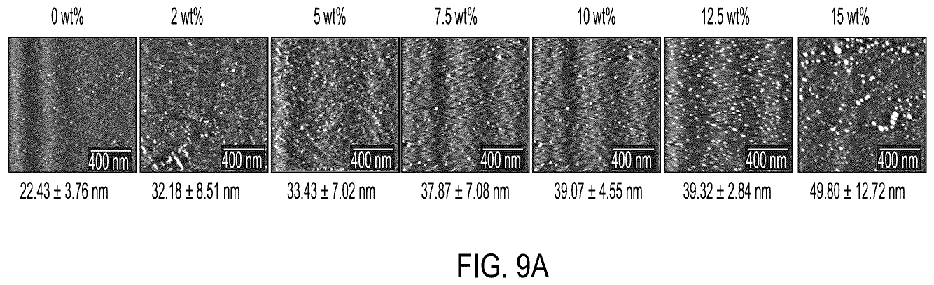

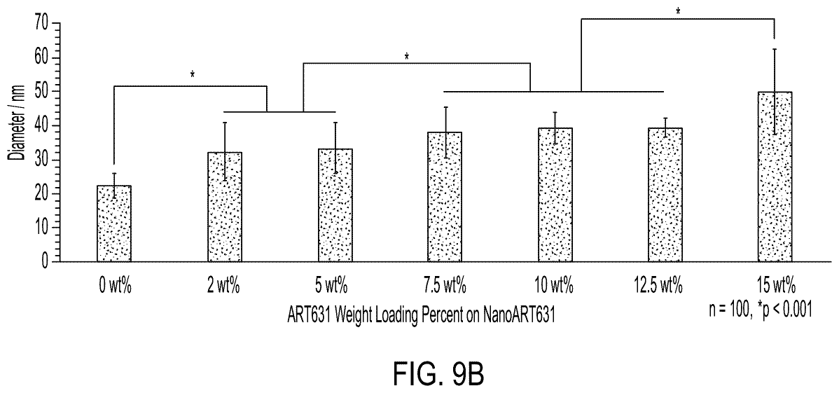

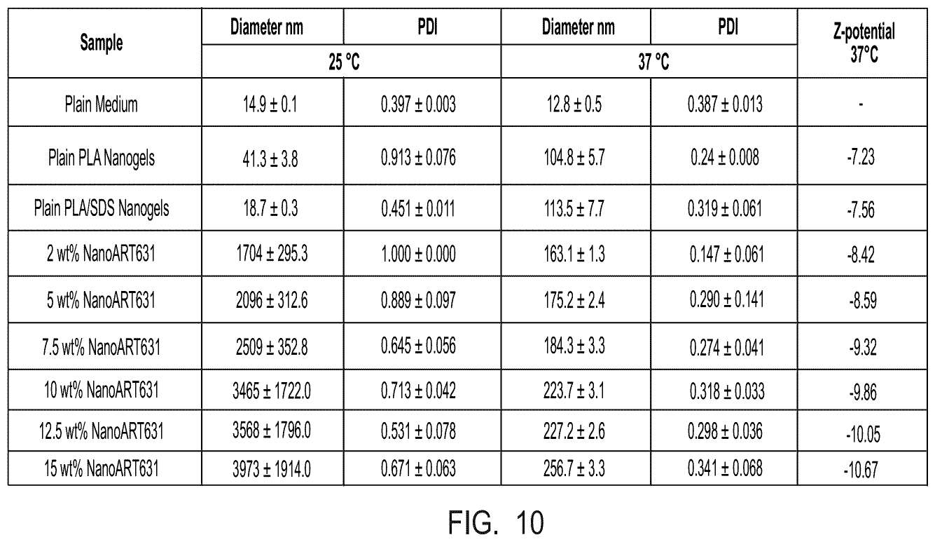

The zeta potential values of NanoART631 are all negative, indicating that the nanogels can be stable in water. The absolute value of zeta potential increases with increasing the ART631 loading content, probably due to contribution of the ether and endoperoxide groups of ART631. The AFM measurement results in FIG. 9 show that the particle sizes of the nanogels at 37° C. increase with increasing the ART631 loading content from 0 to 15 wt %, agreeing with the DLS results (FIG. 10). The particle sizes of the nanogels obtained from the AFM calculation are smaller than those obtained from the DLS measurements. The reasons for this may be that the particles cannot fully expend due to the surface restriction of the AFM substrate, and/or the particles measured by DLS are aggregates.

The particle diameters also were measured in RPMI-1640 cell culture medium (FIG. 10) to examine how the culture medium can affect the nanogel particle size. At 25° C., the particle sizes of plain nanogels with and without 0.5 mM SDS become 5-10 times smaller when the solvent is changed from DI water to RPMI-1640 medium (41.31±3.79 vs. 225.6±0.86 for plain PLA nanogels; 18.74±0.31 vs. 221.9±0.72 nm for plain PLA/SDS nanogels) due to the salting out effect of the salts in RPMI-1640 medium. With the incorporation of 2 wt % ART631 into the nanogels, the particle sizes increase 90 times (1704.0±295.3 vs. 18.74 ±0.31 nm). The size increases with increasing the loading of ART631 and reaches 3973±1914 nm, 212 times higher than the plain nanogels due to the hydrophobicity of ART631 and salting out effect of the medium. When the temperature is increased from 25 to 37° C., the particle sizes of the two plain nanogels increase as the nanogels are thermos-responsive and become more hydrophobic with increasing temperature. For the nanogels containing ART631, the particle sizes decrease with increasing temperature from 25 to 37° C. due to the thermos-shrinking response of PNIPAAM of the nanogels. The sizes of the ART631-loaded nanogels in RPMI-1640 medium (FIG. 10) are larger than the corresponding sizes in water (FIG. 8), especially at 25° C. Without wishing to be bound by theory, the reason is probably due to the salting out effect of the medium on the polyelectrolyte nanogels causing the aggregation of the nanogels, which is more pronounced at 25° C. than at 37° C. as the nanogels swell more at 25° C. than at 37° C. FIG. 10 shows that the nanogels have a PDI smaller than 0.35 at 37° C. The PDI values of the nanogels at 25° C. are higher than those at 37° C., probably because of the higher density of the nanogels at 37° C. due to thermos-shrinkage which mask the contribution of the components in the medium to the scattered light intensity. The zeta potential values of NanoART631 in RPMI-1640 medium are all negative indicating that the nanogels can be stable in RPMI-1640 medium even though their absolute values are slightly lower than those in water (FIG. 8).

### Example 6. In Vitro Release of ART631 from Nanogels

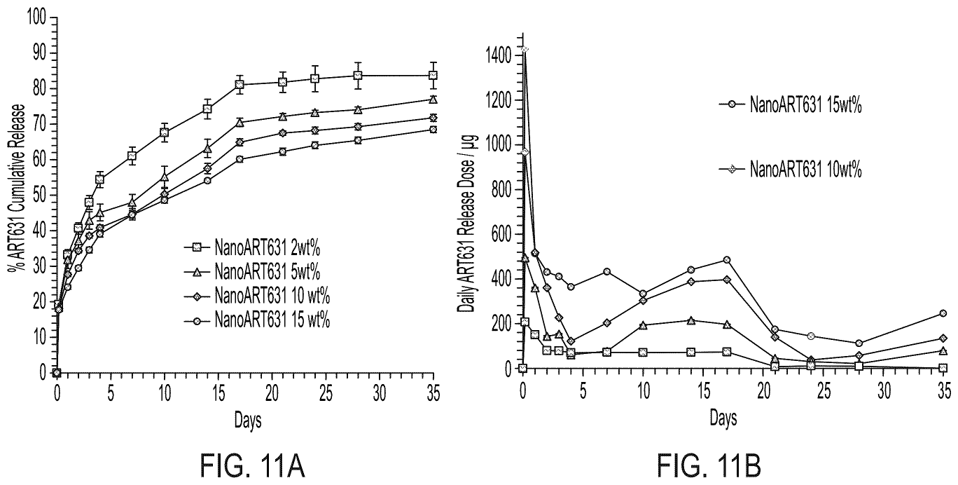

NanoART631 containing 2, 5, 10 and 15 wt % ART631 were dispersed in 1 mL DI water inside a release device with molecular weight cut off 50 kDa at 53.7 mg/mL. The release device was immersed in 20 mL DI water in a 50 mL conical centrifuge tube, and the release was carried out in an incubator shaker at 70 rpm and 37° C. At selected times, the release device was transferred to a new DI water release media maintained at 37° C. The amount of ART631 released was measured using 1H NMR CP800 MHZ (n=4). See FIG. 11, which shows that NanoART631 containing 2, 5, 10 and 15 wt % ART631 can sustain the release of ART631 for at least 35 days. The higher the amount of ART631 loaded in the nanogels, the slower is the ART631 released from the nanogels.

### Example 7. Cell Culturing

The MOLM14 AML cell line, MV4;11, and THP1 cell lines were cultured and incubated in RPMI 1640 medium with 10% FBS at 37° C. and air containing 5% CO₂. When the cells reached about 70% confluency, the cells were split with a density of 40,000 cells per cm². Adult Retinal Pigment Epithelial (ARPE-19) cells were grown in Dulbecco's Modified Eagle Medium (DMEM)/F12 (1:1) with 10% FBS and 1% Penicillin-Streptomycin (Pen-Strep). Dental Pulp Stem Cells (DPSCs) were grown in DMEM with glutamine and 10% FBS and 1% Pen-Strep. A Hemolysis assay kit was purchased from Creative Biolabs™.

### Example 8. In Vitro Cytotoxicity and IC₅₀

The cytotoxicity of plain nanogels, plain SDS nanogels and 2, 5, 10 and 15 wt % ART631-loaded NanoART631 were assessed using thiazolyl blue tetrazolium bromide (MTT) and WST1 assays against MOLM14 human acute myeloid cells, MV4;11 cells, THP1 cells, normal adult retinal pigment epithelium-19 (ARPE-19) cells, and dental pulp stem cells (DPSCs). In brief, 150 μL of ARPE-19 cells or DPSCs were seeded in each well of 96-well plates at 15,000 cells/well density. After 24 hours, 30 μL of solution was removed from each well and replaced by 30 μL of medium containing different concentrations of NanoART631. After 24 hours, 50 μL of 5 mg/mL MTT were added to every well and incubated at 37° C. for 4 hours. For the MOLM14 AML cell line, cells were seeded at a density of 50,000 cells/well and the incubation time of the cells with the samples was 48 hours before the MTT test. Similar steps were used for the 1 to 5-day incubation experiment against the MOLM14 cells, but with a 10,000 cells/well seeding density in 96-well plates. Pure ART631, ART838, 5 and 10 wt % NanoART631 IC₅₀ were determined by adding 5e⁻⁶, 5e⁻⁵, 5e⁻⁴, 6.25e⁻⁴, 0.001, 0.0025, 0.005, 0.01, 0.05, 0.1, 0.2, 0.5, 1, 5, 10 mg·mL-1 of the formulation to MOLM14 AML cells. While the 2 wt % NanoART631 IC₅₀ was determined by adding 5e⁻⁶, 5e⁻⁵, 5e⁻⁴, 0.005, 0.006, 0.007, 0.008, 0.009, 0.01, 0.05, 0.1, 0.2, 0.5, 1, 5, 10 mg/mL of the formulation to MOLM14 AML cells. Absorbance was read at 570 nm using a BioTek™ cytation 5 microplate reader for all experiments after removing all the wells contents and then dissolving the formazan crystals in 150 μL of DMSO. The cytotoxicity of plain nanogels and 2, 5, 10 and 15 wt % loaded NanoART631 to MOLM14, MV4;11 and THP1 cells was assessed using WST1 assay after 48 hours incubation at a 20,000 cells/well seeding density in 96-well plates.

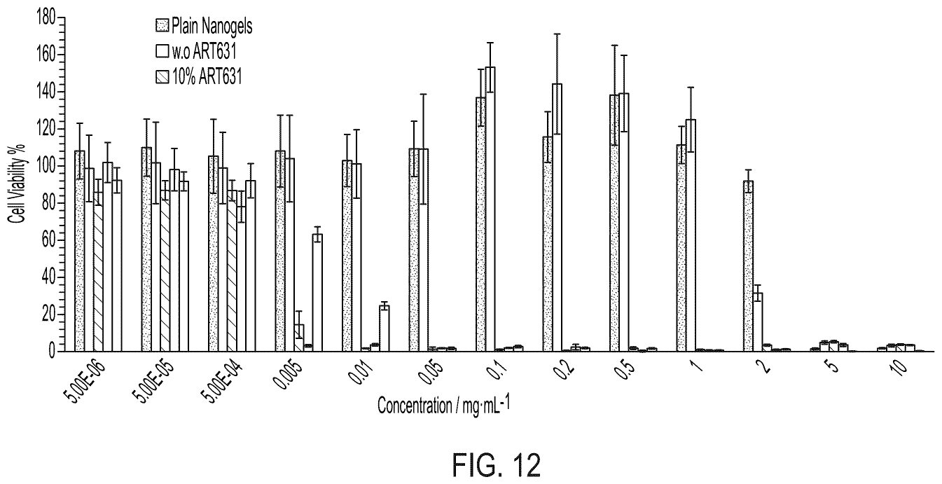

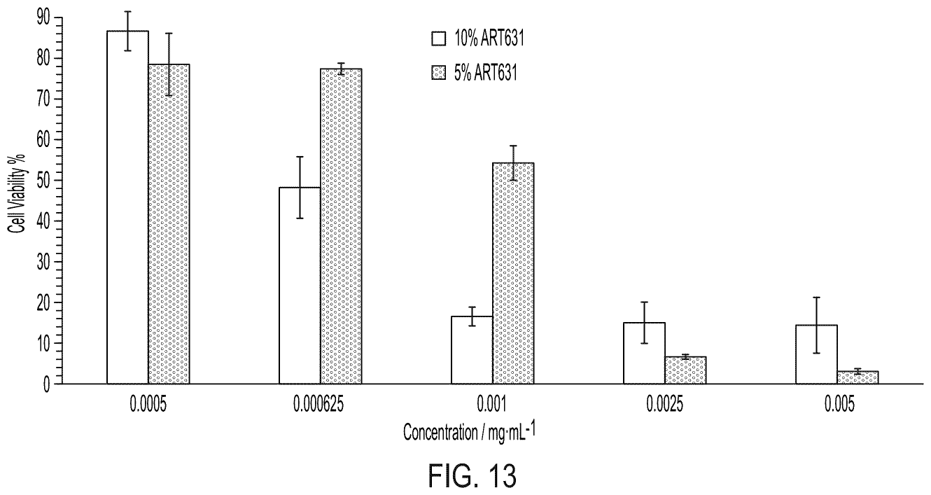

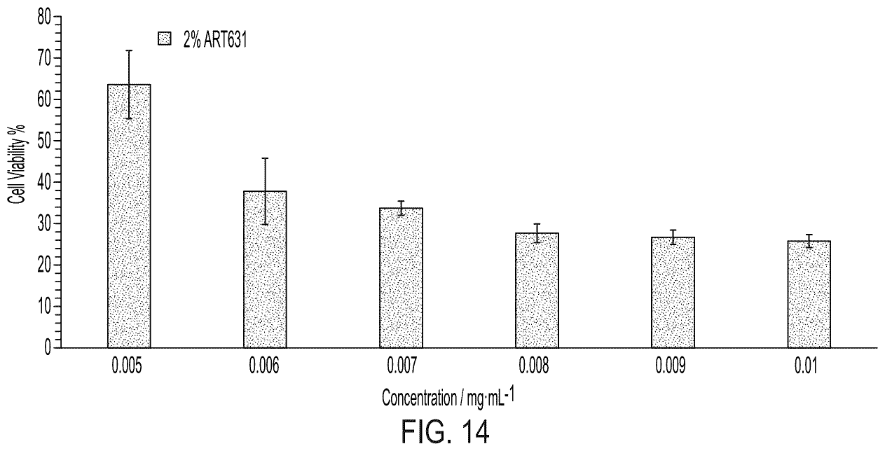

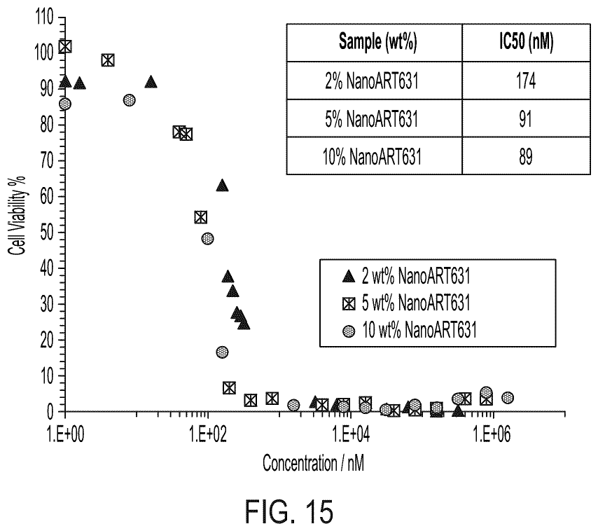

FIG. 12 through FIG. 15 show that plain nanogels and plain SDS nanogels are not toxic to MOLM14 AML cells at concentrations up to 2 mg/mL and 1 mg/mL, respectively, by MTT assay. In contrast, 2 wt % ART631-loaded nanogels killed 62.19% of the cells at 0.006 mg/mL nanogel concentration (190.17 nM ART631 concentration) and about 98% at 0.05 mg/mL nanogel concentration (1584.8 nM ART631); 5 wt % ART631-loaded nanogels killed 51.74% of the cells at 6.25×10⁻⁴ mg/mL (99.05 nM of ART631) and about 97% of the cells at 0.005 mg/mL nanogel concentration (396.2 nM of ART631); and 10 wt % ART631-loaded nanogels killed 45.74% of the cells at 0.001 mg/mL (79.24 Nm of ART631) concentrations and about 98% of the cells at 0.01 mg/mL nanogels concentration (1584.8 nM ART631). The IC₅₀ values of 2, 5 and 10 wt % ART631-loaded nanogels are 174, 91, and 89 nM, respectively (see FIG. 15), suggesting that the ART631-loaded nanogels are very potent in killing MOLM14 AML cells and the effectiveness of killing increases with increasing the loading of ART631 in the nanogels from 2 to 10 wt %.

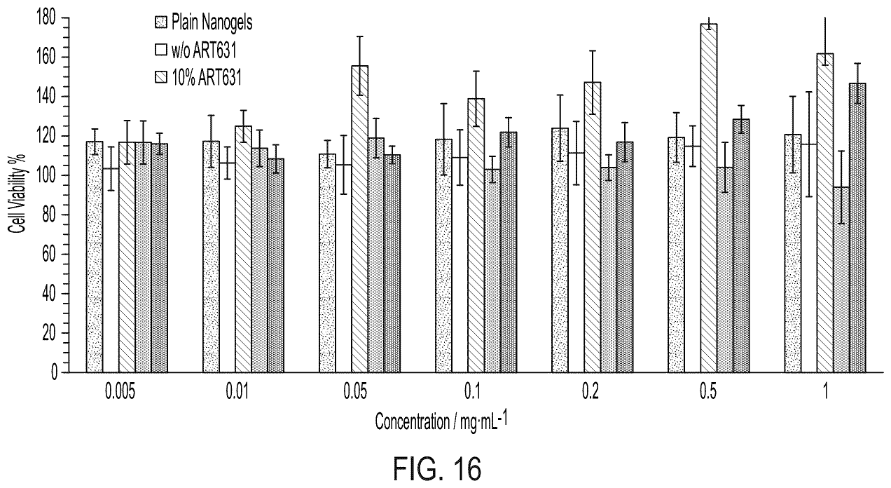

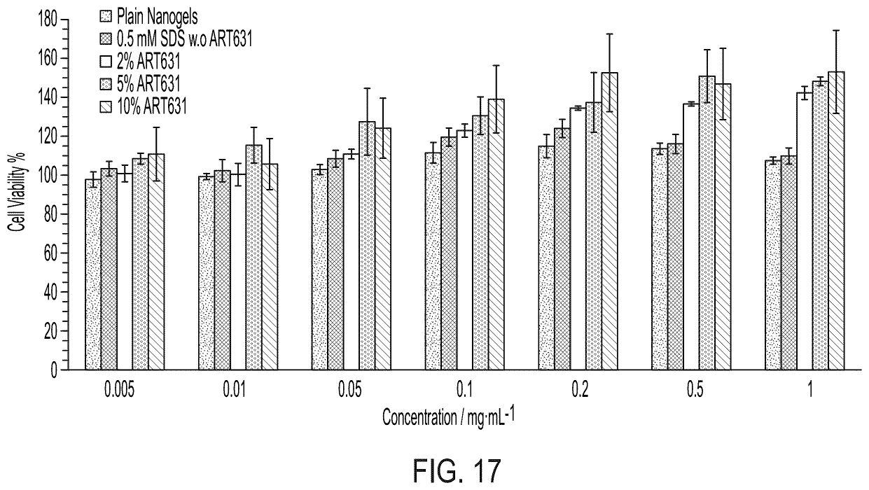

To demonstrate that ART-631-loaded nanogels are specific and selective in killing leukemic cells but safe to normal cells, the cytotoxicity of the nanogels to two normal cell lines ARPE-19 cells and DPSCs was tested using an MTT assay. FIG. 16 and FIG. 17 show that the nanogels loaded with and without ART631 are not toxic to ARPE-19 cells and DPSCs at concentration up to at least 1 mg/mL after 24 hours of incubation.

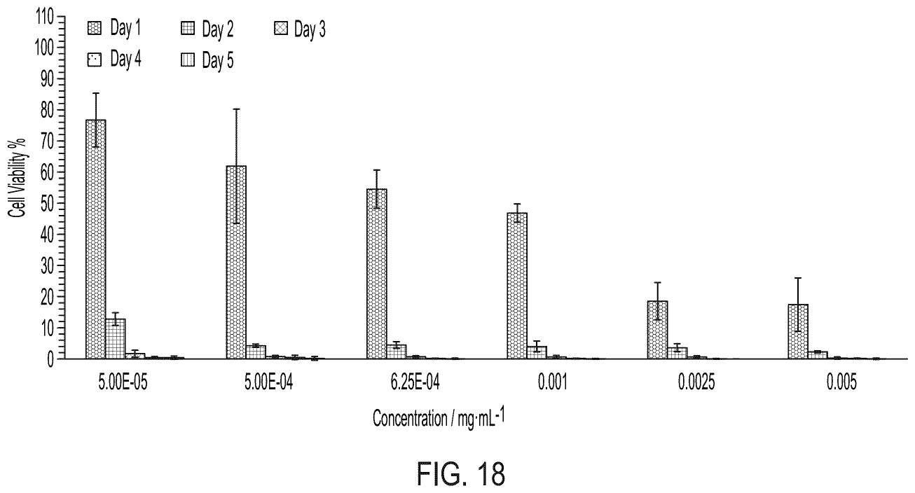

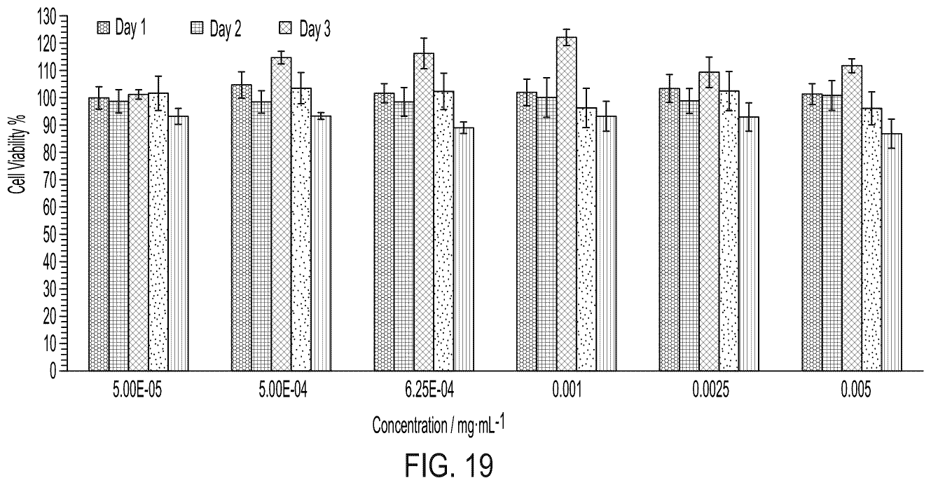

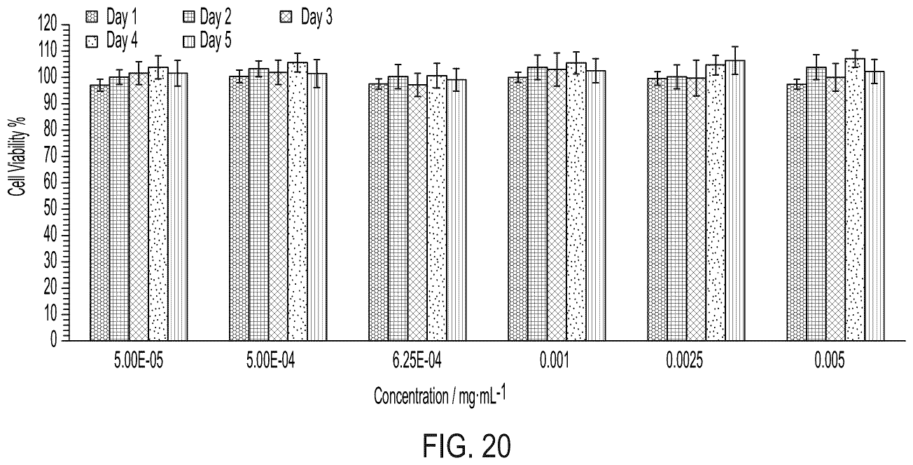

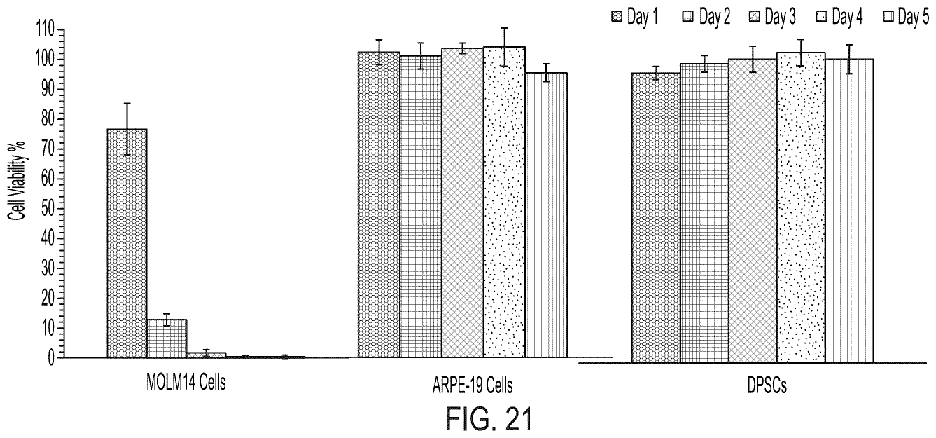

To assess the time-dependent effectiveness of NanoART631 in killing leukemic cells, the cytotoxicity of 5 wt % ART631-loaded nanogels to MOLM14 AML cells, ARPE-19 cells and DPSCs were tested by MTT assay daily for a period of 5 days. For MOLM14 AML cells, a strong cytotoxic effect of NanoART631 to the cells starts on day 2 at all concentrations tested in the range of 5e⁻⁵ to 0.005 mg/mL, and about 98% of the cells are dead on day 3 (see FIG. 18 and FIG. 21). In contrast, both AREP-19 cells (FIG. 19 and FIG. 21) and DPSCs (FIG. 20 and FIG. 21) are 100% viable through the 5 days of study course.

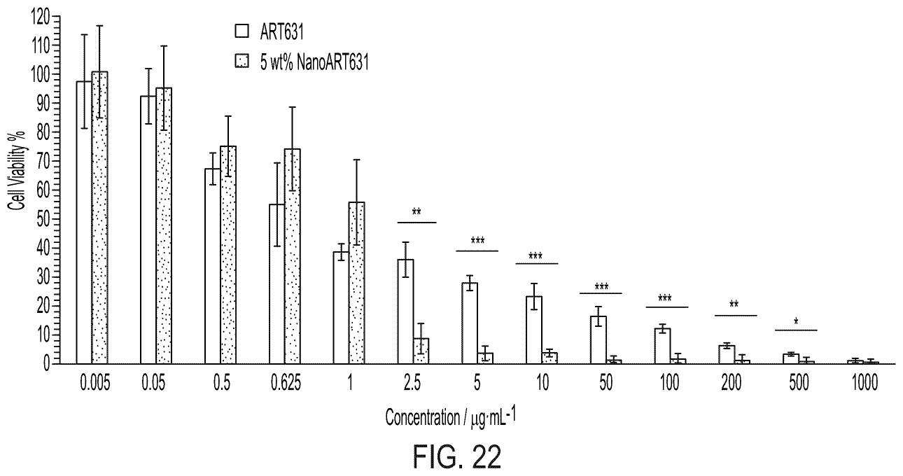

To study the effectiveness of NanoART631 compared to drug alone in killing leukemic cells, the cytotoxicity of 5 wt % ART631-loaded nanogels, ART631 and ART838 to MOLM14 AML cells was tested as a function of concentration by using MTT assay. FIG. 22 shows that ART631 and ART838 drugs alone are almost equally cytotoxic to the MOLM14 cells in the concentration range of 5 e⁻⁶ to 10 mg/mL. After ART631 is loaded into the nanogels, the cytotoxic effect does not change when the concentration is lower than 0.001 mg/mL. However, when the concentration is 0.0025 mg/mL or higher, NanoART631 are more effective in killing MOLM14 cells than ART631 and ART838 drugs alone. For example, NanoART631 kill 91% MOLM14 cells while ART631 and ART838 kill only 67% and 64%, respectively at concentration 0.0025 mg/mL, and the concentration needs to go up to more than 0.05 mg/mL in order for the two drugs to kill 90% cells. The reason may be related to the ability of the nanogels to penetrate through the cell membrane to provide more drug inside the cells for more effective anti-leukemia effect.

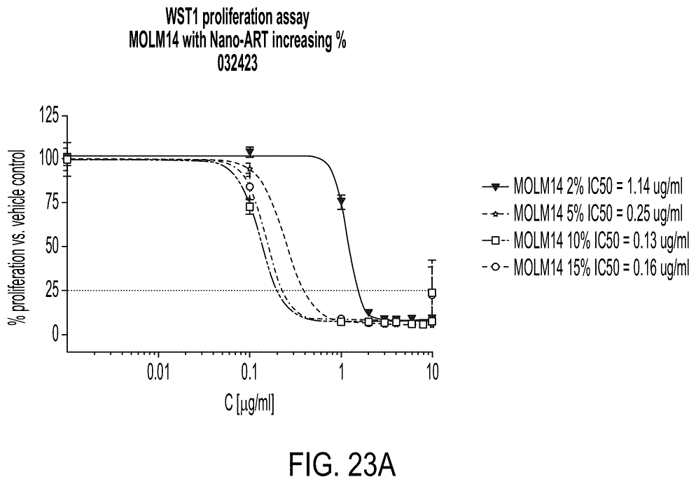

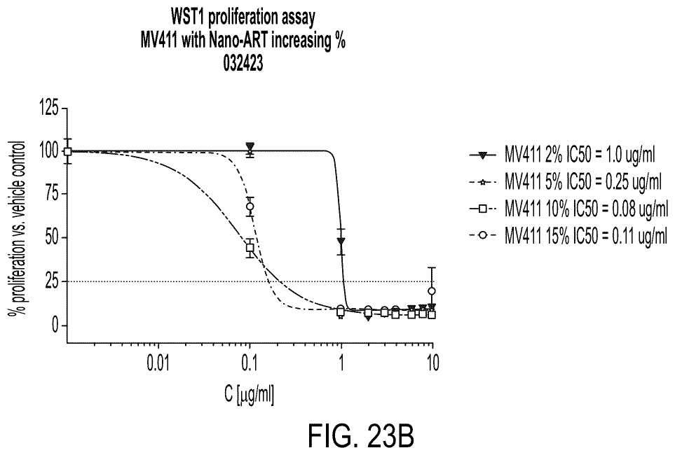

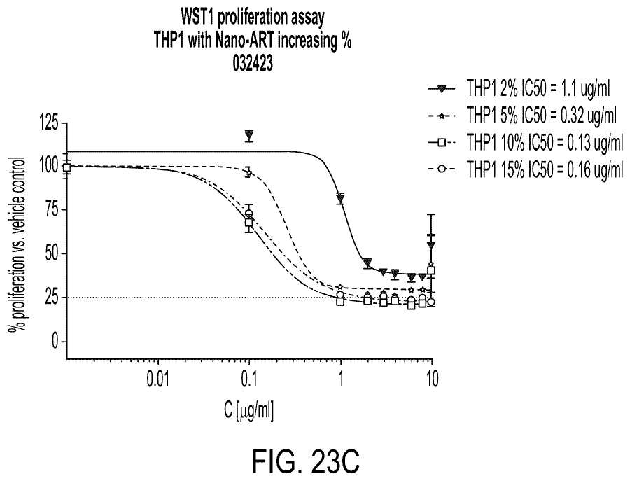

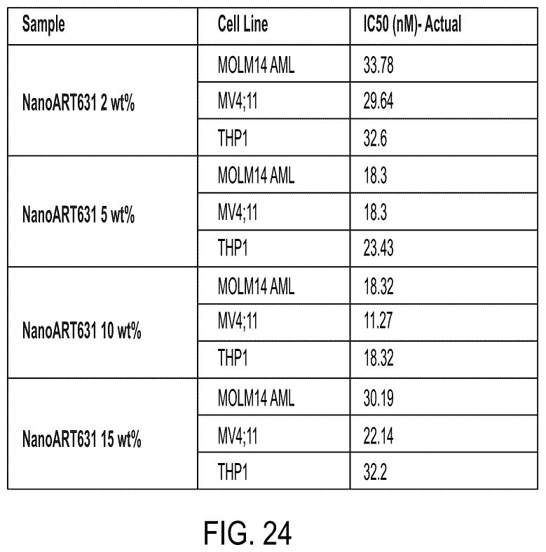

To evaluate the cytotoxicity of NanoART631 to other leukemic cells besides MOLM14 cells, MV4;11 and THP1 cells were tested by WST1 assay. FIG. 23 shows that all the NanoART631 loaded with 2, 5 10, and 15 wt % ART631 are very potent in killing all the three cell lines MOLM14, MV4;11 and THP1 cells after 48 hours incubation at a 20,000 cells/well seeding density in 96-well plates with IC₅₀ concentration lower than 35 nM. See FIG. 24.

### Example 9. In Vivo Toxicology

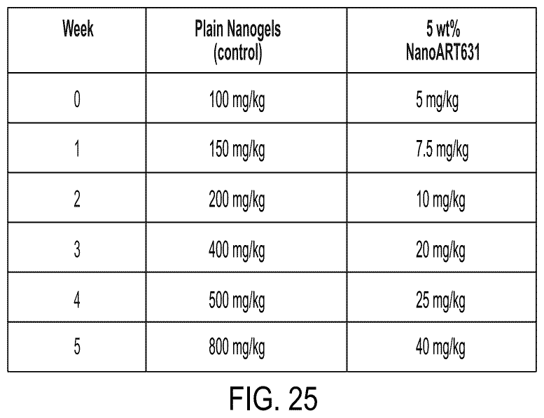

The maximum tolerated doses (MTD) of 0.5 mM SDS plain nanogels and NanoART631 loaded with 5 wt % ART631 were determined after single intravenous (IV) injections in nonobese diabetic/severe combined immunodeficiency gamma (NSG) female mice (n=3). The mice were given single dose every week of increased concentrations as shown in FIG. 25. MTD was defined as 20% body weight loss within 7 days after each injection. The MTD of NanoART631 was 500 mg/kg (containing ˜25 mg/kg ART631). Empty nanoparticles (without ART631) had no clinical toxicity to mice at the same and higher IV dose up to at least 800 mg/kg.

The maximum tolerated doses (MTD) of 0.5 mM SDS plain nanogels and NanoART631 loaded with 5 wt % ART631 were determined after single intravenous (IV) injections in nonobese diabetic/severe combined immunodeficiency gamma (NSG) female mice (n=3). The mice were given single dose every week of increased concentrations as shown in FIG. 25. MTD was defined as 20% body weight loss within 7 days after each injection. The MTD of NanoART631 was 500 mg/kg (containing ˜25 mg/kg ART631). Empty nanoparticles (without ART631) had no clinical toxicity to mice at the same and higher IV dose up to at least 800 mg/kg.

### REFERENCES

All references listed below and throughout the specification are hereby incorporated by reference in their entirety.
1. Moses BS. Blood Adv. 2021; 5:711-721.
2. Efferth T. Int J Oncol. 2001; 18:767-773.
3. Mott B T. Bioorg & Med Chem. 2013; 21:3702-07.
4. Fox J M. Oncotarget. 2016; 7:7268-7276.
5. Janagam D R. Adv Biosys. 2017; 1:1700097.
6. Frediansyah, A. Clin.Epidem.Gl.Health 2021; 9:90-98
7. Zhao, M. Eur J Pharmacol. 2021; 890:173646
8. Beigel, J. H. N Engl J Med 2020; 383:1813-1826
9. Yan, V. C. ACS Med. Chem. 2020; 11:1361-1366
10. Janagam, DR. Pharmaceutics. 2016; 8
11. WHO Coronavirus Dashboard 2021; covid19.who.int
12. Coronavirus (COVID-19) Vaccinations 2021; ourworldindata.org/covid-vaccinations?country=OWID_WRL
14. Frediansyah, A. Clin. Epidem.Gl.Health 2021; 9:90-98
15. Beigel, J. H. N Engl J Med 2020; 383:1813-1826 [6] Yan, V. C. ACS Med. Chem. 2020; 11:1361-1366
16. U.S. Pat. No. 9,487,538.
17. Sung H. et al. ACS Journals 2021; 71:209-249.
18. Cancer Stat Facts: Leukemia 2021; https://seer.cancer.gov/statfacts/html/leuks.html
19. Financial Burden of Cancer Care 2021; https://progressreport.cancer.gov/after/economic_burden
20. Posner, G. H. and Mott, B. T. (2014). Two-carbon linked artemisinin-derived trioxane dimers. U.S. Pat. No. 9,487,538.
21. Huang X, Misra G P, Vaish A, Flanagan J M, Sutermaster B and Lowe T L*. Novel Nanogels with Both Thermoresponsive and Hydrolytically Degradable Properties. Macromolecules 41 (22): 8339-8345, 2008.

1. A polymerized nanogel pharmaceutical composition, comprising:
(a) a hydrophobic drug;
(b) optionally sodium dodecyl sulfate (SDS); and
(c) a biodegradable nanogel composition comprising
(i) hydrolyzable macromer with two or more double bonds;
(ii) a monomer; and
(iii) an initiator,
wherein the macromer, the monomer, and the hydrolysable crosslinker are reacted with an initiator to form a biodegradable nanogel in the presence of the hydrophobic drug.

2. The composition of claim 1 wherein the polymerized nanogel pharmaceutical composition has a size of about 1 nm to 1000 nm.

3. The composition of claim 1 wherein the polymerized nanogel pharmaceutical composition has a size of about 1 to 600 nm.

4. The composition of claim 1 wherein the polymerized nanogel pharmaceutical composition has a size of about 10 to about 350 nm.

5. The composition of claim 1 wherein the hydrophobic drug contains one or more double bonds.

6. The composition of claim 5 wherein the double bond is a C═C double bond.

7. The composition of claim 1 wherein the hydrophobic drug is selected from the group consisting of artemisinin, an artemisinin derivative, ART631, artemisinin conjugated with other anticancer pharmacophores, 3-carbon-linked artemisinin-derived dimer (3C-ART), 2-carbon-linked dimeric artemisinin-derived analogs, osimertinib, sunitinib, quinine, lumefantrine, stiripentol, glecaprevir, cyclosporin, voclosporin, naloxone, betulinic acid, bevirimat, derivatives, dienestrol, neuroprotection, prostaglandin, unsaturated fatty acids, rilpivirine, polyene antimycotics, or a combination thereof.

8. The composition of claim 7 wherein the hydrophobic drug is ART631.

9. The composition of claim 1, wherein the hydrolysable macromer is dextran grafted with oligolactate-(2-hydroxyetheyl methacrylate) (Dex-PLA-HEMA), poly-ε-caprolactone-(2-hydroxyethyl methacrylate (Dex-PCL-HEMA), or a hydrolyzable molecule composed of dextran, polylactic acid, polylactic-co-glycolic acid, polyethylene glycol, poly(ethylene oxide)-poly(propylene oxide)-poly(ethylene oxide), poly(ethylene oxide)-co-poly(L-lactic acid), biotinylated poly(ethylene glycol-block-lactic acid), pluronic acid, polaxamer, polyesters, polyamides, poly(amino acid), polyurethane, polyorthoesters, polyanhydrides, polyethylene terephthalate, polycarbonates, polyfumarates, polycyanoacrylates, poly(alkylcyanoacrylate), polyphosphazenes, polyphosphoesters, or poly(bis(p-carboxyphenoxy) propane-sebacic acid), or a combination thereof, wherein the hydrolysable molecule contains one or more double bonds.

10. The composition of claim 1 wherein the monomer is selected from the group consisting of N-isopropylacrylamide, N-alkylacrylamide, N-n-propylacrylamide, N-isopropylmethacrylamide, or any combination thereof.

11. The composition of claim 1, wherein the initiator is selected from the group consisting of 2-hydroxy-4′-(2-hydroxyethoxy)-2-methylpropiophenone, 2,2′-azobis [2-(2-imidazolin-2-yl) propane] dihydrochloride, 1-hydroxycyclohexylphenyl-ketone, 2-hydroxy-2-methyl-1-phenylpropanone, 2,2-dimethoxy-1,2-diphenyl-ethan-1-one, 2-(4-Methylbenzyl)-2-(dimethylamino)-1-(4-morpholinophenyl) butan-1-one, alpha hydroxy ketones, phosphine oxides, benzophenone, thioxanthones, 2,2-dimethoxy-2-phenylacetophenone, isopropyl thioxanthone, 2-ethylhexyl-(4-N,N-dimethyl amino)benzoate, ethyl-4-(dimethylamino)benzoate, peroxides, benzoyl peroxide, molecular oxygen, azobisisobutyronitrile, camphorquinone, eosin Y, triethanolamine, 1-vinyl-2-pyrrolidinone, or a combination thereof.

12. The nanogel pharmaceutical composition of claim 5 wherein the hydrophobic drug is released from the nanogel for at least 50 days.

13. The nanogel pharmaceutical composition of claim 5 wherein the hydrophobic drug is released from the nanogel for at least 60 days.

14. The nanogel pharmaceutical composition of claim 5 wherein the hydrophobic drug is released from the nanogel for at least 90 days.

15. The method of drug delivery to a subject in need thereof comprising administering to the subject the nanogel pharmaceutical composition of claim 1.

16. The method of drug delivery of claim 15 wherein the subject is suffering from a disease or conditions selected from the group consisting of cancer, malaria, fungus, infection, inflammation, seizure, stroke, depression, hepatitis C, diabetes, diabetic retinopathy, age-related macular degeneration, glaucoma, dry eye, Alzheimer's disease, Parkinson's disease, neurological disorders, pain, temporomandibular joint disorders, immune system disorders, opioid overdose or thereof.

17. The method of drug delivery of claim 16 wherein the subject is suffering from cancer.

18. The method of drug delivery of claim 17 wherein the cancer is leukemic, skin, melanoma, lung, bronchus, kidney, liver, breast, oral, head, neck, esophageal, thyroid, eye, retinal, ear, bone, cartilage, fat, muscle, blood vessel, gastrointestinal stromal, intrahepatic bile duct, bladder, colon, rectum, vaginal, prostate, testicular, pancreatic, cervical, uterine, pleural, immune system, glioblastoma, Non-Hodgkin lymphoma, carcinoma-adenocarcinoma, basal cell carcinoma, squamous cell carcinoma and transitional cell carcinoma, sarcoma, lymphoma, myeloma, brain, or spinal cord cancer thereof.

19. The method of drug delivery of claim 18 wherein the cancer is a leukemia.

20. The method of drug delivery of claim 15 wherein the nanogel pharmaceutical composition is administered by injection.

21. A method of increasing the solubility and effective pharmacologic half-life of a double bond-containing hydrophobic drug compound using a nanogel pharmaceutical composition of claim 1.

22. A method of making a nanogel pharmaceutical composition of claim 1, comprising:
(a) dissolving a double bond containing hydrophobic drug compound in an organic solvent;
(b) adding the hydrophobic drug to solution a solution of water optionally also containing SDS;
(c) dissolving a macromer selected from the group consisting of dextran-polylactic acid-2-hydroxyethyl-methacrylate, dextran grafted oligolactate-(2-hydroxyethyl methacrylate), and poly-ε-caprolactone-(2-hydroxyethyl methacrylate (Dex-PCL-HEMA) macromer, in water also optionally containing SDS to form a solution;
(d) combining the macromer solution and NIPAAM solution to the drug compound solution;
(e) adding 2-hydroxy-4′-(2-hydroxyethyl)-2-methyl propiophenone to the combined mixture in water;
(f) incubating the mixture in an oil bath with nitrogen purging; and
(g) during nitrogen purging, curing the mixture solution by exposure to UV light for at a wavelength of 320 nm to 500 nm for about 2 minutes to about 5 hours.
